(19) United States
(12) Patent Application Publication
(10) Pub. No.: US 2015/0267258 A1
(43) Pub. Date: Sep. 24, 2015

(54) BIOMARKERS FOR DETERMINING EFFECTIVE RESPONSE OF TREATMENTS OF HEPATOCELLULAR CARCINOMA (HCC) PATIENTS

(71) Applicant: BAYER PHARMA AKTIENGESELLSCHAFT, Berlin (DE)

(72) Inventors: Heiko Krissel, Berlin (DE); Florian Pühler, Wellesley, MA (US); Michael Jeffers, Ridgewood, NJ (US)

(21) Appl. No.: 14/404,648

(22) PCT Filed: May 27, 2013
(86) PCT No.: PCT/EP2013/060854

§ 371 Date: Dec. 1, 2014

(30) Foreign Application Priority Data
May 31, 2012 (EP) 12170199.9

(51) Int. Cl. C12Q 1/68; A61K 31/4412; A61K 31/18

(52) U.S. Cl. CPC C12Q 1/6886; A61K 31/18; A61K 31/4412; C12Q 2600/156; C12Q 2600/106

(57) ABSTRACT

This invention is directed to the use of one or more biomarkers defined as KRAS or NRAS gene for predicting the pharmaceutical efficacy or clinical response of MEK protein kinase inhibitor and/or Sorafenib or Regorafenib to be administered to a Hepatocellular carcinoma (HCC) patient. Further the invention is directed to in-vitro methods for identifying mutated-type KRAS or NRAS gene in HCC patient and kits thereof.

### FIELD OF THE INVENTION

This invention is directed to the use of one or more biomarkers defined as KRAS or NRAS gene for predicting the pharmaceutical efficacy or clinical response of MEK protein kinase inhibitor and/or Sorafenib or Regorafenib to be administered to a Hepatocellular carcinoma (HCC) patient. Further the invention is directed to in-vitro methods for identifying mutated-type KRAS or NRAS gene in HCC patient and kits thereof.

### BACKGROUND OF THE INVENTION

Oncogenes—genes that contribute to the production of cancers—are generally mutated forms of certain normal cellular genes (“proto-oncogenes”). Oncogenes often encode abnormal versions of signal pathway components, such as receptor tyrosine kinases, serine-threonine kinases, or downstream signaling molecules. The central downstream signaling molecules are the Ras proteins, which are anchored on the inner surfaces of cytoplasmic membranes, and which hydrolyze bound guanosine triphosphate (GTP) to guanosine diphosphate (GDP). When activated by a growth factor, growth factor receptors initiate a chain of reactions that leads to the activation of guanine nucleotide exchange activity on Ras. Ras alternates between an active “on” state with a bound GTP (hereafter “Ras.GTP”) and an inactive “off state with a bound GDP. The active “on” state, Ras.GTP, binds to and activates proteins that control the growth and differentiation of cells.

For example, in the “mitogen-activated protein kinase (MAP kinase) cascade,” Ras.GTP leads to the activation of a cascade of serine/threonine kinases. One of several groups of kinases known to require a Ras.GTP for their own activation is the Raf family. The Raf proteins activate “MEK1” and “MEK2,” abbreviations for mitogen-activated ERK-activating kinases (where ERK is extracellular signal-regulated protein kinase, another designation for MAPK). MEK1 and MEK2 are dual-function serine/threonine and tyrosine protein kinases and are also known as MAP kinase kinases. Thus, Ras.GTP activates Raf, which activates MEK1 and MEK2, which activate MAP kinase (MAPK). Activation of MAP kinase by mitogens appears to be essential for proliferation, and constitutive activation of this kinase is sufficient to induce cellular transformation. Blockade of downstream Ras signaling, as by use of a dominant negative Raf-1 protein, can completely inhibit mitogenesis, whether induced from cell surface receptors or from oncogenic Ras mutants.

The interaction of Raf and Ras is a key regulatory step in the control of cell proliferation. To date, no substrates of MEK other than MAPK have been identified; however, recent reports indicate that MEK may also be activated by other upstream signal proteins such as MEK kinase or MEKK1 and PKC. Activated MAPK translocates and accumulates in the nucleus, where it can phosphorylate and activate transcription factors such as Elk-1 and Sap1a, leading to the enhanced expression of genes such as that for c-fos.

Once activated, Raf and other kinases phosphorylate MEK on two neighboring serine residues, S218 and S222 in the case of MEK1. These phosphorylations are required for activation of MEK as a kinase. In turn, MEK phosphorylates MAP kinase on two residues separated by a single amino acid: a tyrosine, Y185 and a threonine, T183.

MEK appears to associate strongly with MAP kinase prior to phosphorylating it, suggesting that phosphorylation of MAP kinase by MEK may require a prior strong interaction between the two proteins. Two factors—MEK's unusual specificity and its requirement for a strong interaction with MAP kinase prior to phosphorylation—suggest that MEK's mechanism of action may differ sufficiently from the mechanisms of other protein kinases as to allow for selective inhibitors of MEK. Possibly, such inhibitors would operate through allosteric mechanisms rather than through the more usual mechanism involving blockage of an ATP binding site.

Thus, MEK1, MEK2 and Raf are validated and accepted targets for anti-proliferative.

RAS genes are involved in human tumors. Oncogenic mutant RAS proteins are resistant to downregulation by GAP-mediated hydrolysis of bound GPT. The RAS subfamily includes at least 21 members like HRAS, KRAS, NRAS, RRAS. Mutations in RAS gene play a direct role in causing cancer (Amy Young et al. Advances in Cancer Research, 2009). Large number of mutation of the RAS proteins were identified and quantified in several tumors (Yuliya Pylayeva-Gupta et al. Nature Review—Cancers, vol 11, November 2011, p 761 and Antoine E. Karmoud et al. Nature Review—Cancers, vol 9, July 2008, p 517).

### MEK Protein Kinase Inhibitor s:

Several examples of 1-substituted-2(p-substituted-phenylamino)-aryl inhibitors of MEK have been reported. U.S. Pat. Nos. 6,440,966 and 6,750,217 and corresponding publication WO 00/42003 described carboxylic and hydroxamic acid esters and N-substituted amide derivatives of sulfonamide-substituted-2(4-iodophenylamino)-benzoic acid esters and N-substituted benzamides as functioning as MEK inhibitors. The sulfonamide may also be N-substituted.

U.S. Pat. No. 6,545,030 and corresponding publication WO 00/42029 describe MEK inhibitors that are 1-heterocyclyl-2(4-iodophenylamino)-benzene, where the heterocycle is a five-membered nitrogen-containing ring such as pyrazole, triazole, oxazole, isoxazole, and isoxazolinone. The more recent U.S. Patent Publication 2005/004186 describes related compounds in which the 4-iodo substituent of the '030 patent is replaced by a very broad genus of moieties including alkyl, alkoxy, acyloxy, alkenyl, carbamoyl, carbamoylalkyl, carboxyl, carboxylalkyl, N-acylsulfonamido, and others.

U.S. Pat. No. 6,469,004 and corresponding publication WO 00/42022 describe carboxylic and hydroxamic acid esters of a group of heterocyclo-condensed phenylene compounds, i.e., benzimidazoles, benzooxazoles, benzothiazoles, benzothiadiazoles, quinazolines, etc. The heterocycles are 7-F-6-(4-iodo-phenylamino)-5-carboxylic acid esters, carboxylic acid amides or hydroxamic acid esters. More recent publication U.S. 2005/0026970 described similar compounds in which the 4-iodo substituent was replaced by a very broad genus of structures. Related compounds are described in patent publications WO 03/077855, WO 03/77914 and US 2005/0554701. Further examples of 2-(4-iodophenylamino)-phenylhydroxamic acid esters which are reported to be useful as MEK inhibitors can be found in WO 2005/028426.

Patent Publication WO 02/06213 and corresponding U.S. application Ser. No. 10/333,399 (U.S. 2004/0054172) describe hydroxy-substituted acid esters of 1-oxamic acid-2(4-halophenylamino)-3,4-difluorobenzene. U.S. Pat. No. 6,891,066 and corresponding publication WO 03/62191 describe similar compounds wherein the 4-halo substituent is replaced by a very broad genus of structures. Among the substituents in the 4-position were methyl, ethyl, ethynyl, and 2-hydroxyethyl. Specific related compounds are described in U.S. Pat. No. 6,770,778.

Patent Publication WO 04/083167, published Sep. 30, 2004, (in Japanese) discloses more than two thousand—but provides NMR data for only 400—1-(N-substituted sulfonyl urea)-2(2,4-dihalophenylamino)-3,4-difluorobenzenes and asserts that they useful as MEK inhibitors. Data indicating inhibition of MEK were presented for a subgroup of just twelve. In addition to a secondary or tertiary amine, these twelve compounds all contained one of the following groups: an N, N-disubstituted sulfonyl urea, N-piperazinesulfonamide, N-piperidinesulfonamide or N-pyrrolidinesulfonamide.

Recently, N-(2-arylamino) aryl sulfonamides were described as suitable MEK inhibitors in WO 2007/014011 A2. Those N-(2-arylamino) aryl sulfonamides are new mitogen activated extracellular-signal-regulated kinase (ERK) kinase (MEK) inhibitor that have demonstrated broad anti-tumor activity as a single agent and synergistic activity.

### Sorafenib:

Sorafenib (Nexavar®; Bayer AG, Leverkusen, Germany) is an oral multikinase inhibitor that is able to inhibit several tyrosine kinase receptors involved in angiogenesis and lymphangiogenesis, including vascular endothelial growth factor receptor (VEGFR)-1, VEGFR-2, VEGFR-3, platelet-derived growth factor receptor (PDGFR), Filt-3, c-Kit and RET (Wilhelm et al, 2006; Wilhelm et al, 2004). In addition, sorafenib inhibits the Ras/Raf/mitogen-activated protein (MAP)/extracellular-signal regulated kinase (ERK) kinase (MEK) [or mitogen activated protein kinase (MAPK)] pathway, which has been implicated in cell proliferation, differentiation, and survival in a variety of solid tumours and leukaemic cell lines (Sebolt-Leopold & Herrera 2004; Roberts & Der 2007; Wilhelm et al, 2004; Yu et al, 2005). The cell death promoting effects of sorafenib may vary among cell lines, and they seem to involve cytostatic and cytotoxic mechanisms that have only partially been elucidated. In lymphoma cells, sorafenib exposure down-regulates the anti-apoptotic protein myeloid cell leukaemia-1 (Mcl-1), a Bel-2 family member that has been implicated in cell survival. Mcl-1 is overexpressed in several lymphomas and may confer resistance to apoptotic stimuli exerted by most cytotoxic drugs (Rahmani et al, 2007; Cory et al, 2003; Cho-Vega et al, 2004; Yu et al, 2005). Additionally, sorafenib-induced inhibition of the ERK pathway might result in Bel-X_{L} down-regulation, thus mimicking rituximab-mediated effects on CD20-positive NHL cell lines (Jazirehi et al, 2004). Recently, sorafenib were found to be effective in lung cancer (Edward S. Kim et al., American Association for Cancer Research, Cancer Discovery, 2011; 1(1) OF43).

### Regorafenib:

Regorafenib (US20050038080 and WO2005009961) is an oral multi-kinase inhibitor which targets angiogenic, stromal and oncogenic receptor tyrosine kinase (RTK). Regorafenib shows anti-angiogenic activity due to its dual targeted VEGFR2-TIE2 tyrosine kinase inhibition. It is currently being studied as a potential treatment option in multiple tumor types.

Regorafenib has been shown to increase the overall survival of patients with metastatic colorectal cancer.

Hepatocellular carcinoma (HCC) is the sixth most common neoplasm and the third cause of cancer-related death. More than 75% of cases occur in the Asia-Pacific region, largely in association with chronic hepatitis B virus (HBV) infection. More than 50% of cases of HCC occur in China alone, and an estimated 360000 patients residing in East Asian countries, including China, Japan, Korea, and Taiwan, die from this disease each year.

The prognosis for patients with HCC remains dismal. The overall 5-year survival rate of HCC patients is only 9%, which is only slightly better than the 4% recorded for those diagnosed 3 decades ago. Even for those with HCC confined to the liver, the 5-year survival rate is only 19%, and it falls to 7% for those with regional spread and 3.4% for those with distant disease.

Despite above mentioned advances for treatment of cancer, a major challenge in cancer treatment is the selection of patients for specific treatment regimens based on genetic markers, namely biomarkers, in order to optimize treatment outcome.

In other words, it would be helpful to know which patients are able to positively respond to an intended treatment consisting of the administration to a Hepatocellular carcinoma (HCC) patient of MEK protein kinase inhibitor and/or Sorafenib or Regorafenib, wherein the MEK inhibitor is a N-(2-arylamino) aryl sulfonamide.

Indeed, it was surprisingly found that the use of a specific biomarker namely RAS gene is suitable for selecting Hepatocellular carcinoma (HCC) patients responding positively to administration of MEK protein kinase inhibitor and/or Sorafenib or Regorafenib.

Therefore, there is a need for diagnostic test, methods and tools using RAS gene as biomarkers that are suitable for providing predictive information about patient's responses.

### SUMMARY OF THE INVENTION

In a First aspect, the invention is directed to the use of one or more biomarkers defined as mutated RAS for predicting the pharmaceutical efficacy or clinical response of a combination comprising a MEK protein kinase inhibitor and Sorafenib or Regorafenib to be administered to a HCC patient.

In a Second aspect, the invention is directed to the use of one or more biomarkers defined as mutated RAS for predicting the pharmaceutical efficacy or clinical response of at least one MEK protein kinase inhibitor to be administered to a HCC patient.

In a Third aspect, the invention is directed to the use of one or more biomarkers defined as mutated RAS for predicting the pharmaceutical efficacy or clinical response of Sorafenib or Regorafenib to be administered to a HCC patient.

In a Fourth aspect, the invention is directed to an in-vitro method comprising the step of
Identifying mutated-type RAS gene and/or protein in a test sample obtained from a HCC patient, characterized in that the method is for predicting the pharmaceutical efficacy or clinical response of a combination comprising a MEK protein kinase inhibitors and/or Sorafenib or Regorafenib to be administered to a HCC patient.

In a Fifth aspect, the invention is directed to a kit.

In a sixth aspect, the invention is directed to the use of a compound of formula A as defined herein, for the preparation of a medicament for the treatment of hepatocellular carcinoma in a patient possessing a mutated KRAS, NRAS or HRAS gene.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

It was surprisingly found that the presence of a mutated RAS biomarker correlates with the treatment efficacy of a MEK protein kinase inhibitor and/or Sorafenib or Regorafenib administered to a HCC patient.

In a First aspect, the invention is directed to the use of one or more biomarkers defined as mutated RAS for predicting the pharmaceutical efficacy or clinical response of a combination comprising a MEK protein kinase inhibitor and Sorafenib or Regorafenib to be administered to a HCC patient.

In one embodiment, the use is directed to one or two biomarkers defined as RAS. Preferably, the use is directed to one biomarker defined as RAS.

In one embodiment, the RAS is referring to gene or protein thereof wherein the RAS gene or RAS protein is selected from KRAS, NRAS or HRAS. Preferably, RAS is KRAS or NRAS. More preferably, RAS is KRAS.

Preferably, the use is directed to KRAS, NRAS or HRAS gene. More preferably, the use is directed to KRAS or NRAS gene.

RAS protein is a protein corresponding to the transduction of one RAS gene.

In one embodiment, the use is directed to one biomarker defined as mutated KRAS or NRAS gene or protein thereof for predicting the pharmaceutical efficacy or clinical response of a combination of the MEK protein kinase inhibitor and Sorafenib or Regorafenib to be administered to a HCC patient.

In one embodiment, the use is directed to a combination of a MEK protein kinase inhibitor and Sorafenib or Regorafenib.

Mutated KRAS, NRSA and HRAS genes or proteins are well known in the literatures tumors (Yuliya Pylayeva-Gupta et al. Nature Review—Cancers, vol 11, November 2011, p 761 and Antoine E. Karmoud et al. Nature Review—Cancers, vol 9, July 2008, p 517).

Mutated KRAS and NRSA genes are, preferably, defined as in table 1.

TABLE 1
KRAS and NRAS gene and protein mutations

| | | Nucleotide | | | Amino Acid |
|---|---|---|---|---|---|
| Gene | Exon | Position | Change | Report | Change |
| KRAS | 1 | 34 | G > A | g34a | G12S |
| | | 34 | G > C | g34c | G12R |
| | | 34 | G > T | g34t | G12C |
| | | 35 | G > A | g35a | G12O |
| | | 35 | G > C | g35c | G12A |
| | | 35 | G > T | g35t | G12V |
| | | 38 | G > A | g38a | G13O |
| KRAS | 2 | 183 | A > C | a183c | O61H |
| KRAS | 3 | 436 | G > A | g436a | A146T |
| NRAS | 3 | 181 | C > A | c181a | O61K |
| | | 182 | A > G | a182g | O61R |
| | | 182 | A > T | a182t | O61L |
| | | 183 | A > T | a183t | O61H |

Predicting the pharmaceutical efficacy or clinical response means that HCC patient responding positively (reduction of tumor or stable tumor growth) to treatment can be differentiated from HCC patient not responding to treatment.

HCC patient means patient suffering from Hepatocellular carcinoma.

In one embodiment, the MEK protein kinase inhibitor is selected from the group of CI-1040 (PD184352), GSK1120212, PD-0325901, PD-98059, PD-184161, PD-0318088, PD-184386, PD-171984, PD-170611, PD-177168, PD-184352, ARRY-438162, AZD6244/ARRY-886, AZD 8330, XL518, UO125, UO126, SL 327, quercetin, or a pharmaceutically acceptable salt, solvate, polymorph, ester, and tautomer thereof.

In other embodiment, the MEK protein kinase inhibitor is a compound of formula A, or a pharmaceutically acceptable salt, solvate, poilymorph, ester, amide, tautomer or prodrug thereof:

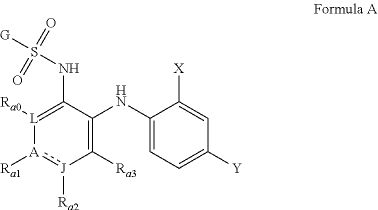

wherein
G is G₁, G₂, R₁ₐ, R_{1b}, R_{1c}, R_{1d}, R₁ₑ, Ar₁, Ar₂ or Ar₃;
Rₐ₀, R₁ and R₂ are independently selected from H, halogen, cyano, cyanomethyl, nitro, difluoromethoxy, difluoromethoxy, trifluoromethyl, azido, amino, alkylamino, dialkylamino, C₂R₅, OR₅, —O—(CO)—R₅, —O—C(O)—N(R₅)₂, —NR₅C(O)NR₆R₇, —SR₅, NHC(O)R₅, —NHSO₂R₅, SO₂N(R₅)₂, C1-C6 alkyl, C1-C4 alkoxy, C3-C6 cycloalkyl, C2-C6 alkenyl, C2-C6 alkynyl, aryl, alkylaryl, arylalkyl, and heterocyclic;
   each R₅ is selected from H, lower alkyl, substituted lower alkyl, aryl, or substituted aryl, and NR₇R₆; wherein each R₆ and R₇ is independently selected from hydrogen or lower alkyl; wherein
   said alkyl, cycloalkyl, alkenyl, aryl, alkylaryl, arylalkyl, heterocyclic and alkynyl groups are optionally substituted with 1-3 substituents selected independently from halogen, OH, CN, cyanomethyl, nitro, phenyl, difluoromethoxy, difluoromethoxy, and trifluoromethyl;
   said C1-C6 alkyl and C1-C4 alkoxy groups are optionally substituted with OCH₃ or OCH₂CH₃;
Rₐ₁ is H, C₁-C₆ alkyl, C₃-C₆ cycloalkyl, C₂-C₆ alkenyl, C₅-C₆ cycloalkenyl or C₂-C₆ alkynyl;
   wherein each alkyl, cycloalkyl, alkenyl, cycloalkenyl or alkynyl group is optionally substituted with 1-3 substituents selected independently from halogen, hydroxy, C₁-C₄ alky, C₁-C₄ alkoxy, cyano, cyanomethyl, nitro, azido, trifluoromethyl difluoromethoxy and phenyl, and
   one or two ring carbon atoms of said C₃-C₆ cycloalkyl groups are optionally replaced with, independently, O, N, or S; or
Rₐ₁ is a 5 or 6-atom heterocyclic group, which group may be saturated, unsaturated, or aromatic, containing 1-5 heteroatoms selected independently from O, N, and S, which heterocyclic group is optionally substituted with 1-3 substituents selected independently from halogen, hydroxy, C₁-C₄ alky, C₁-C₄ alkoxy, cyano, cyanomethyl, nitro, azido, trifluoromethyl difluoromethoxy and phenyl;
Rₐ₂ is H, halogen, F, or oxo; or
Rₐ₁ and Rₐ₂, taken together, are -Q(R₂)—U(R₁)=D-
Rₐ₃ is H, halogen, hydroxy, azido, cyano, cyanomethy, C₁-C₆ alkyl, C₃-C₆ cycloalkyl, C₂-C₆ alkenyl, C₅-C₆ cycloalkenyl or C₂-C₆ alkynyl, wherein each alkyl, cycloalkyl, alkenyl cycloalkenyl or alkynyl group is optionally substituted with 1-3 substituents selected independently from halogen, hydroxy, C₁-C₄ alkoxy, cyano, cyanomethyl, nitro,
[symbol] is a single or a double bond;
X and Y are independently selected from F, I, Br, Cl, CF₃, C1-C3 alkyl, C2-C3 alkenyl, C2-C3 alkynyl, cyclopropyl, phenyl, pyridyl, pyrazolyl, OMe, OEt, or SMe, or Het, where Het is a 5- to 10-membered mono- or bicyclic heterocyclic group, which group is saturated, olefinic, or aromatic, containing 1-5 ring heteroatoms selected independently from N, 0, and S; where
   all said phenyl or Het groups are optionally substituted with F, Cl, Br, I, acetyl, methyl, CN, NO₂, CO₂H, C₁-C₃ alkyl, C₁-C₃ alkoxy, C₁-C₃ alkyl-C(═O)—, C₁-C₃ alkyl-C(═S)—, C₁-C₃ alkoxy-C(═S)—, C₁-C₃ alkyl-C(═O)O—, C₁-C₃ alkyl-O—(C═O)—, C₁-C₃ alkyl-C(═O)NH—, C₁-C₃ alkyl-C(═NH)NH—, C₁-C₃ alkyl-NH—(C═O)—, di-C₁-C₃ alkyl-N—(C═O)—, C₁-C₃ alkyl-C(═O)N(C₁-C₃ alkyl)-, C₁-C₃ alkyl-S(═O)₂NH— or trifluoromethyl;
   all said methyl, ethyl, C1-C3 alkyl, and cyclopropyl groups of X and Y are optionally substituted with OH;
   all said phenyl, pyridyl, pyrazolyl groups of Y are optionally substituted with halogen, acetyl, methyl, and trifluoromethyl; and all said methyl groups of X and Y are optionally substituted with one, two, or three F atoms;
A, D, J, L, Q, U are independently selected from C, CH, —NH, N, O, and —N(CH₃)—;
G₁ is C₁-C₆ alkyl optionally substituted with one amino, C₁-C₃ alkylamino, or dialkylamino group, said dialkylamino group comprising two C₁-C₄ alkyl groups which may be identical or non-identical; or
G₁ is a C₃-C₅ diamino alkyl group;
G₂ is a 5- or 6-membered ring, which is saturated, unsaturated, or aromatic, containing 1-3 ring heteroatoms selected independently from N, O, and S, optionally substituted with 1-3 substituents selected independently from F, Cl, OH, O(C₁-C₃ alkyl), OCH₃, OCH₂CH₃, CH₃C(═O)NH, CH₃C(═O)O, CN, CF₃, and a 5-membered aromatic heterocyclic group containing 1-4 ring heteroatoms selected independently from N, O, and S;
R₁ₐ is methyl, cyclopropoxy or C1-C4 alkoxy; wherein
   the methyl is optionally substituted with OH, 1-3 fluorine atoms or 1-3 chlorine atoms;
   the C1-C4 alkyl moieties of said C1-C4 alkoxy are optionally substituted with one hydroxy or methoxy group; and
   all C2-C4 alkyl groups within said C1-C4 alkoxy are optionally further substituted with a second OH group;
R_{1b} is CH(CH₃)—C1-3 alkyl or C3-C6 cycloalkyl, said CH₃, alkyl, and cycloalkyl groups optionally substituted with 1-3 substituents selected independently from F, Cl, Br, I, OH, C1-C4 alkoxy and CN;
R_{1c} is (CH₂)ₙOₘR′, where
   m is 0 or 1;
   n is 0, 1, 2, or 3;
   R′ is C1-C6 alkyl, optionally substituted with 1-3 substituents selected independently from F, Cl, OH, OCH₃, OCH₂CH₃, and C3-C6 cycloalkyl;
R_{1d} is C(A′)(A″)(B)— wherein
   B, A′, and A″ are, independently, H, substituted or unsubstituted C1-6 alkyl, substituted or unsubstituted C2-6 alkenyl, or
   A′ and A″, together with the carbon atom to which they are attached, form a substituted or unsubstituted 3- to 6-member saturated ring;
R₁ₑ is benzyl or 2-phenyl ethyl, in which the phenyl group is optionally substituted

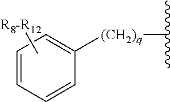

where
q is 1 or 2;
R₈ and R₉ are, independently, H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl;
R₁₀ is H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl, nitro, acetamido, amidinyl, cyano, carbamoyl, methylcarbamoyl, dimethylcarbamoyl, 1,3,4-oxadiazol-2-yl, 5-methyl-1,3,4-5 oxadiazolyl, 1,3,4-thiadiazolyl, 5-methyl-1,3,4-thiadiazol-1H-tetrazolyl, N-morpholinyl carbonylamino, N-morpholinylsulfonyl or N-pyrrolidinylcarbonylamino;
R₁₁ and R₁₂ are, independently, H, F, Cl, or methyl;
Ar₁ is

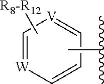

where
W and V are, independently, N, CR₈ or CR₉;
R₈, R₉ and R₁₀ are, independently, H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl, nitro, acetamido, amidinyl, cyano, carbamoyl, methylcarbamoyl, dimethylcarbamoyl, 1,3,4-oxadiazol-2-yl, 5-methyl-1,3,4-oxadiazol, 1,3,4-thiadiazol, 5-methyl-1,3,4-thiadiazol, 1H-tetrazolyl, N-morpholinylcarbonylamino, N-morpholinylsulfonyl and N-pyrrolidinylcarbonylamino;
R₁₁ and R₁₂ are, independently, H, F, Cl or methyl;
Ar₂ is

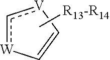

where
the dashed line represents a double bond which may be located formally either between V and the carbon between W and V, or between W and the carbon between W and V;
W is —S—, —O— or —N═, wherein
   when W is —O— or —S—, V is —CH═, —CCl═ or —N═; and
   when W is —N═, V is CH, CCl, N or —NCH₃—;
R₁₃ and R₁₄ are, independently, H, methoxycarbonyl, methylcarbamoyl, acetamido, acetyl, methyl, ethyl, trifluoromethyl or halogen;
Ar₃ is

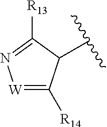

where
W is —NH—, —NCH₃— or —O—; and
R₁₃ and R₁₄ are, independently, H, F, Cl, or methyl.

In one embodiment, the MEK protein kinase inhibitors is selected from the group consisting of a compound of formula I

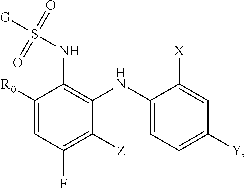

a compound of formula II,

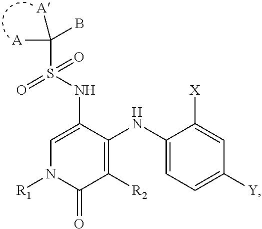

and a compound of formula III,

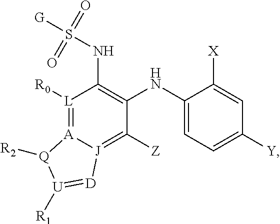

or a pharmaceutically acceptable salt, solvate, polymorph, ester, amide, or tautomer thereof.

In one embodiment, the MEK protein kinase inhibitors is

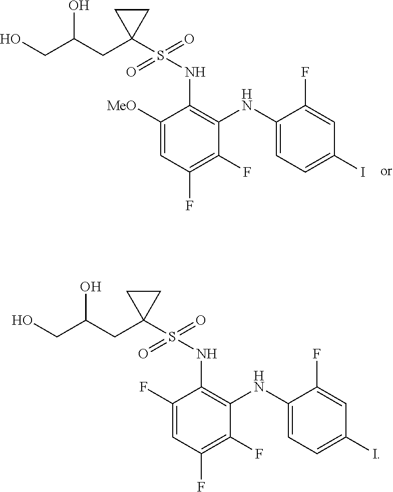 [SMILES: COc1cc(F)c(F)c(Nc2ccc(I)cc2F)c1NS(=O)(=O)C1(CC(O)CO)CC1 ; "or" ; O=S(=O)(Nc1c(F)cc(F)c(F)c1Nc1ccc(I)cc1F)C1(CC(O)CO)CC1]

In one embodiment, the MEK protein kinase inhibitors is

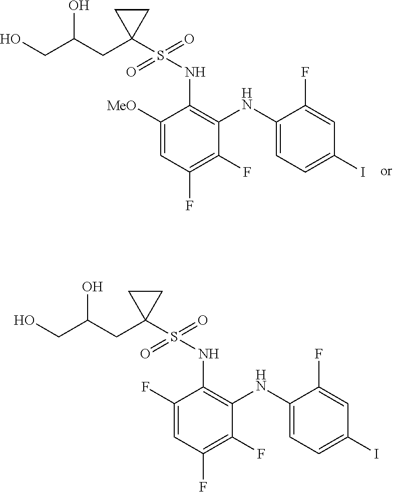 [SMILES: COc1cc(F)c(F)c(Nc2ccc(I)cc2F)c1NS(=O)(=O)C1(CC(O)CO)CC1 ; "or" ; O=S(=O)(Nc1c(F)cc(F)c(F)c1Nc1ccc(I)cc1F)C1(CC(O)CO)CC1]

where the 2-OH carbon is in the R configuration. In one embodiment, the MEK protein kinase inhibitors

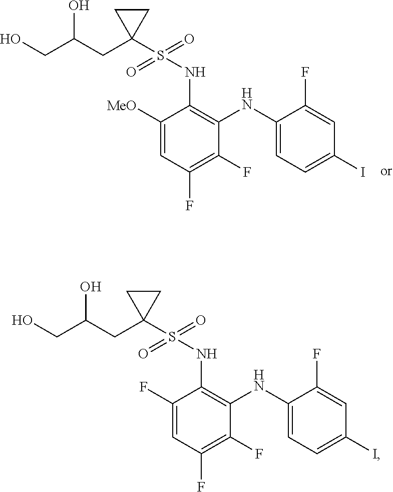 [SMILES: COc1cc(F)c(F)c(Nc2ccc(I)cc2F)c1NS(=O)(=O)C1(CC(O)CO)CC1 ; "or" ; O=S(=O)(Nc1c(F)cc(F)c(F)c1Nc1ccc(I)cc1F)C1(CC(O)CO)CC1]

where the 2-OH carbon is in the S configuration.

In one embodiment, the MEK protein kinase inhibitors is

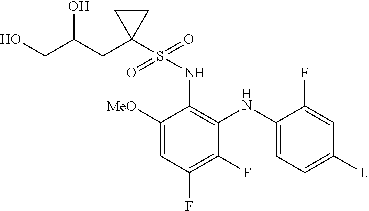 [SMILES: COc1cc(F)c(F)c(Nc2ccc(I)cc2F)c1NS(=O)(=O)C1(CC(O)CO)CC1]

In one embodiment, the MEK protein kinase inhibitors is

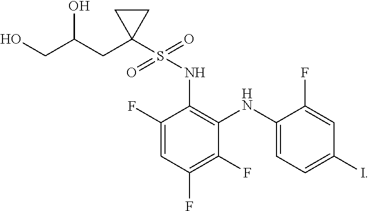 [SMILES: O=S(=O)(Nc1c(F)cc(F)c(F)c1Nc1ccc(I)cc1F)C1(CC(O)CO)CC1]

Sorafenib has the chemical name 4-[4-[[4-chloro-3-(trifluoromethyl)phenyl]carbamoylamino]phenoxy]-N-methyl-pyridine-2-carboxamide and the following chemical structure:

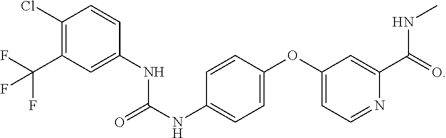 [SMILES: CNC(=O)c1cc(Oc2ccc(NC(=O)Nc3ccc(Cl)c(C(F)(F)F)c3)cc2)ccn1]

Regorafenib has the chemical name 4-[4-({[4-Chloro-3-(trifluoromethyl)phenyl]carbamoyl}amino)-3-fluorophenoxy]-N-methylpyridine-2-carboxamide and the following chemical structure:

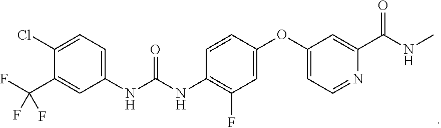 [SMILES: CNC(=O)c1cc(Oc2ccc(NC(=O)Nc3ccc(Cl)c(C(F)(F)F)c3)c(F)c2)ccn1]

In one embodiment, the use is directed to one biomarker defined as mutated KRAS or NRAS gene or protein thereof for predicting the pharmaceutical efficacy or clinical response of a combination of the MEK protein kinase inhibitor and Sorafenib to be administered to a HCC patient

wherein the MEK protein kinase inhibitor is

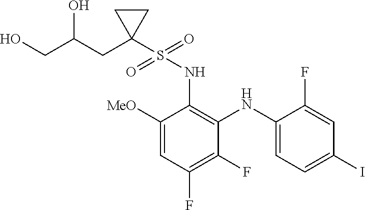 [SMILES: COc1cc(F)c(F)c(Nc2ccc(I)cc2F)c1NS(=O)(=O)C1(CC(O)CO)CC1]

where the 2-OH carbon is in the S configuration.

In one embodiment, the use is directed to one biomarker defined as mutated KRAS or NRAS gene or protein thereof for predicting the pharmaceutical efficacy or clinical response of a combination of the MEK protein kinase inhibitor and Regorafenib to be administered to a HCC patient

wherein the MEK protein kinase inhibitor is

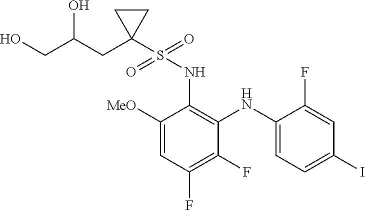 [SMILES: COc1cc(F)c(F)c(Nc2ccc(I)cc2F)c1NS(=O)(=O)C1(CC(O)CO)CC1]

where the 2-OH carbon is in the S configuration

In a Second aspect, the invention is directed to the use of one or more biomarkers defined as mutated RAS for predicting the pharmaceutical efficacy or clinical response of at least one MEK protein kinase inhibitor to be administered to a HCC patient.

In one embodiment, the use is directed to one biomarker defined as mutated KRAS or NRAS gene or protein thereof for predicting the pharmaceutical efficacy or clinical response of one MEK protein kinase inhibitor to be administered to a HCC patient.

Preferably, the MEK protein kinase inhibitor is

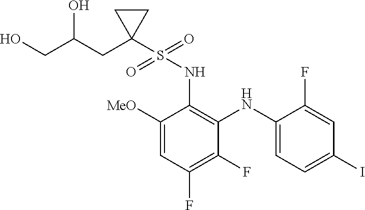 [SMILES: COc1cc(F)c(F)c(Nc2ccc(I)cc2F)c1NS(=O)(=O)C1(CC(O)CO)CC1]

where the 2-OH carbon is in the S configuration.

The embodiments of the first aspect are herein included.

In a Third aspect, the invention is directed to the use of one or more biomarkers defined as mutated RAS for predicting the pharmaceutical efficacy or clinical response of Sorafenib or Regorafenib to be administered to a HCC patient. Preferably, the use is directed to Sorafenib.

The embodiments of the first aspect are herein included.

In a Fourth aspect, the invention is directed to an in-vitro method comprising the step of
Identifying mutated-type RAS gene and/or protein in a test sample obtained from a HCC patient, characterized in that the method is for predicting the pharmaceutical efficacy or clinical response of a combination comprising a MEK protein kinase inhibitor and Sorafenib or Regorafenib MEK protein kinase inhibitors and/or Sorafenib or Regorafenib to be administered to a HCC patient.

Identifying means detecting mutated-type RAS gene or protein in a HCC patient. Several methods for detecting mutated-type RAS gene or protein are known and available on the market e.g. Cobas® KRAS Mutation Test marketed by Roche.

Other methods are discussed in following publications:
Diehl F, Li M, He Y, Kinzler K W, Vogelstein B, Dressman D. (2006) BEAMing: single-molecule PCR on micoparticles in water-in-oil Emulsions. Nat Methods. 2006 July; 3(7):551-9 and
Diehl F., Schmidt K., Choti M. A., Romans K., Goodman S., Li M., Thornton K., Agrawal N., Sokoll L., Szabo S. A., Kinzler K. W., Vogelstein B., Diaz L. A. Jr. (2008) Circulating mutant DNA to assess tumor dynamics. Nature Medicine 14, 985-90.

In one embodiment, test sample means blood sample or tissue sample of a HCC patient. Preferably, test sample means blood sample of a HCC patient.

In one embodiment, the in-vitro method comprises additionally the step of comparison of a mutated RAS to a wild type RAS reference.

In one embodiment, the in-vitro method comprising the step of
Identifying mutated-type RAS gene and/or protein in a test sample obtained from a HCC patient, characterized in that the method is for predicting the pharmaceutical efficacy or clinical response of MEK protein kinase inhibitors and Sorafenib to be administered to a HCC patient
   wherein the MEK protein kinase inhibitor is

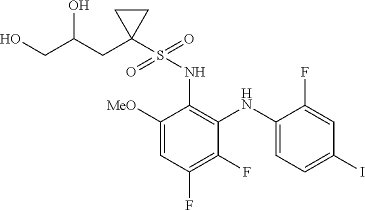 [SMILES: COc1cc(F)c(F)c(Nc2ccc(I)cc2F)c1NS(=O)(=O)C1(CC(O)CO)CC1]

where the 2-OH carbon is in the S configuration.

Preferably, the RAS is referring to gene or protein thereof wherein the RAS gene or protein is selected from KRAS, NRAS or HRAS. Preferably, RAS is KRAS or NRAS. More preferably, RAS is KRAS.

In one embodiment, the in-vitro method comprising the step of
Identifying mutated-type RAS gene and/or protein in a test sample obtained from a HCC patient, characterized in that the method is for predicting the pharmaceutical efficacy or clinical response of MEK protein kinase inhibitors and Regorafenib to be administered to a HCC patient
   wherein the MEK protein kinase inhibitor is

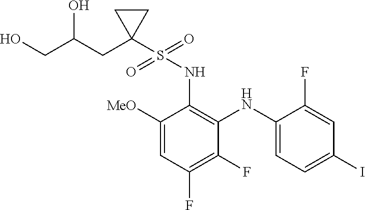 [SMILES: COc1cc(F)c(F)c(Nc2ccc(I)cc2F)c1NS(=O)(=O)C1(CC(O)CO)CC1]

where the 2-OH carbon is in the S configuration.

Preferably, the RAS is referring to gene or protein thereof wherein the RAS gene or protein is selected from KRAS, NRAS or HRAS. Preferably, RAS is KRAS or NRAS. More preferably, RAS is KRAS.

The embodiments of the first aspect are herein included.

In a Fifth aspect, the invention is directed to a kit comprising a suitable means for detecting mutated-type RAS gene or protein, for identifying biomarker defined as a mutated-type RAS,

characterized in that the kit is for predicting the pharmaceutical efficacy or clinical response of a combination comprising a MEK protein kinase inhibitor and Sorafenib or Regorafenib to be administered to a HCC patient.

The embodiments of the first aspect are herein included.

In a sixth aspect, the invention is directed to the use of a compound of formula A as defined herein, for the preparation of a medicament for the treatment of hepatocellular carcinoma in a patient possessing a mutated KRAS, NRAS or HRAS gene.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### Certain Chemical Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting.

Definition of standard chemistry terms may be found in reference works, including Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, —CH₂O— is equivalent to —OCH₂—.

Unless otherwise noted, the use of general chemical terms, such as though not limited to “alkyl,” “amine,” “aryl,” are equivalent to their optionally substituted forms. For example, “alkyl,” as used herein, includes optionally substituted alkyl.

The compounds presented herein may possess one or more stereocenters and each center may exist in the R or S configuration, or combinations thereof. Likewise, the compounds presented herein may possess one or more double bonds and each may exist in the E (trans) or Z (cis) configuration, or combinations thereof. Presentation of one particular stereoisomer, regioisomer, diastereomer, enantiomer or epimer should be understood to include all possible stereoisomers, regioisomers, diastereomers, enantiomers or epimers and mixtures thereof. Thus, the compounds presented herein include all separate configurational stereoisomeric, regioisomeric, diastereomeric, enantiomeric, and epimeric forms as well as the corresponding mixtures thereof. Presentation of one particular chemical structure or chemical name for a compound which contains one or more chiral centers, but which does not designate a particular stereochemistry, should be understood to include all possible stereoisomers, including mixtures of all possible stereoisomers, pure forms or substantially pure forms of one particular stereoisomer and pure forms or substantially pure forms of the alternate stereoisomer. Techniques for inverting or leaving unchanged a particular stereocenter, and those for resolving mixtures of stereoisomers are well known in the art and it is well within the ability of one of skill in the art to choose an appropriate method for a particular situation. See, for example, Furniss et al. (eds.), VOGEL'S ENCYCLOPEDIA OF PRACTICAL ORGANIC CHEMISTRY 5.sup.TH ED., Longman Scientific and Technical Ltd., Essex, 1991, 809-816; and Heller, Acc. Chem. Res. 1990, 23, 128.

The terms “moiety”, “chemical moiety”, “group” and “chemical group”, as used herein refer to a specific segment or functional group of a molecule. Chemical moieties are often recognized chemical entities embedded in or appended to a molecule.

The term “bond” or “single bond” refers to a chemical bond between two atoms, or two moieties when the atoms joined by the bond are considered to be part of larger substructure.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined below. Further, an optionally substituted group may be un-substituted (e.g., —CH2CH3), fully substituted (e.g., —CF2CF3), mono-substituted (e.g., —CH2CH2F) or substituted at a level anywhere in-between fully substituted and mono-substituted (e.g., —CH2CHF2, —CH2CF3, —CF2CH3, —CFHCHF2, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons (except in those instances where macromolecular substituents are clearly intended, e.g., polypeptides, polysaccharides, polyethylene glycols, DNA, RNA and the like).

Unless otherwise noted, the use of general chemical terms, such as though not limited to “alkyl,” “amine,” “aryl,” are unsubstituted.

As used herein, C1-Cx includes C1-C2, C1-C3 . . . C1-Cx. By way of example only, a group designated as “C1-C4” indicates that there are one to four carbon atoms in the moiety, i.e. groups containing 1 carbon atom, 2 carbon atoms, 3 carbon atoms or 4 carbon atoms, as well as the ranges C1-C2 and C1-C3. Thus, by way of example only, “C1-C4 alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “A and A′, together with the carbon atom to which they are attached, form a 3- to 6-member saturated ring”, as used herein, refers to the following structures for compounds of formula I:

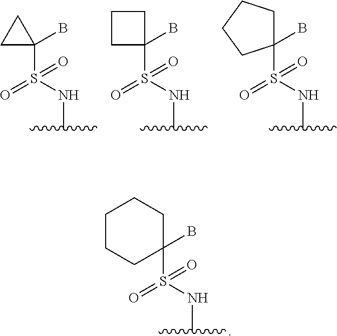

The terms “heteroatom” or “hetero” as used herein, alone or in combination, refer to an atom other than carbon or hydrogen. Heteroatoms are may be independently selected from among oxygen, nitrogen, sulfur, phosphorous, silicon, selenium and tin but are not limited to these atoms. In embodiments in which two or more heteroatoms are present, the two or more heteroatoms can be the same as each another, or some or all of the two or more heteroatoms can each be different from the others.

The term “alkyl” as used herein, alone or in combination, refers to a straight-chain or branched-chain saturated hydrocarbon monoradical having from one to about ten carbon atoms, or one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C1-C6 alkyl” or “C1-6 alkyl”, means that the alkyl group may consist of 1 carbon atom, 2 carbon atoms, 3 carbon atoms, 4 carbon atoms, 5 carbon atoms or 6 carbon atoms. In one embodiment, the “alkyl” is substituted. Unless otherwise indicated, the “alkyl” is unsubstituted.

The term “alkenyl” as used herein, alone or in combination, refers to a straight-chain or branched-chain hydrocarbon monoradical having one or more carbon-carbon double-bonds and having from two to about ten carbon atoms, or two to about six carbon atoms. The group may be in either the cis or trans conformation about the double bond(s), and should be understood to include both isomers. Examples include, but are not limited to ethenyl (—CH═CH2), 1-propenyl (—CH2CH═CH2), isopropenyl [—C(CH3)=CH2], butenyl, 1,3-butadienyl and the like. Whenever it appears herein, a numerical range such as “C2-C6 alkenyl” or “C2-6 alkenyl”, means that the alkenyl group may consist of 2 carbon atoms, 3 carbon atoms, 4 carbon atoms, 5 carbon atoms or 6 carbon atoms. In one embodiment, the “alkenyl” is substituted. Unless otherwise indicated, the “alkenyl” is unsubstituted.

The term “alkynyl” as used herein, alone or in combination, refers to a straight-chain or branched-chain hydrocarbon monoradical having one or more carbon-carbon triple-bonds and having from two to about ten carbon atoms, or from two to about six carbon atoms. Examples include, but are not limited to ethynyl, 2-propynyl, 2-butynyl, 1,3-butadiynyl and the like. Whenever it appears herein, a numerical range such as “C2-C6 alkynyl” or “C2-6 alkynyl”, means that the alkynyl group may consist of 2 carbon atoms, 3 carbon atoms, 4 carbon atoms, 5 carbon atoms or 6 carbon atoms. In one embodiment, the “alkynyl” is substituted. Unless otherwise indicated, the “alkynyl” is unsubstituted.

The terms “heteroalkyl”, “heteroalkenyl” and “heteroalkynyl” as used herein, alone or in combination, refer to alkyl, alkenyl and alkynyl structures respectively, as described above, in which one or more of the skeletal chain carbon atoms (and any associated hydrogen atoms, as appropriate) are each independently replaced with a heteroatom (i.e. an atom other than carbon, such as though not limited to oxygen, nitrogen, sulfur, silicon, phosphorous, tin or combinations thereof), or heteroatomic group such as though not limited to —O—O—, —S—S—, —O—S—, —S—O—, ═N—N═, —N═N—, —N═N—NH—, —P(O)₂—, —O—P(O)₂—, —P(O)₂—O—, —S(O)—, —S(O)₂—, —SnH₂— and the like.

The terms “haloalkyl”, “haloalkenyl” and “haloalkynyl” as used herein, alone or in combination, refer to alkyl, alkenyl and alkynyl groups respectively, as defined above, in which one or more hydrogen atoms is replaced by fluorine, chlorine, bromine or iodine atoms, or combinations thereof. In some embodiments two or more hydrogen atoms may be replaced with halogen atoms that are the same as each another (e.g. difluoromethyl); in other embodiments two or more hydrogen atoms may be replaced with halogen atoms that are not all the same as each other (e.g. 1-chloro-1-fluoro-1-iodoethyl). Non-limiting examples of haloalkyl groups are fluoromethyl, chloromethyl and bromoethyl. A non-limiting example of a haloalkenyl group is bromoethenyl. A non-limiting example of a haloalkynyl group is chloroethynyl.

The term “carbon chain” as used herein, alone or in combination, refers to any alkyl, alkenyl, alkynyl, heteroalkyl, heteroalkenyl or heteroalkynyl group, which is linear, cyclic, or any combination thereof. If the chain is part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining the chain length. If the chain contains heteroatoms as part of the backbone, those atoms are not calculated as part of the carbon chain length.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. Rings can be optionally substituted. Rings can form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “fused” as used herein, alone or in combination, refers to cyclic structures in which two or more rings share one or more bonds.

The term “cycloalkyl” as used herein, alone or in combination, refers to a saturated, hydrocarbon monoradical ring, containing from three to about fifteen ring carbon atoms or from three to about ten ring carbon atoms, though may include additional, non-ring carbon atoms as substituents (e.g. methylcyclopropyl). Whenever it appears herein, a numerical range such as “C3-C6 cycloalkyl” or “C3-6 cycloalkyl”, means that the cycloalkyl group may consist of 3 carbon atoms, 4 carbon atoms, 5 carbon atoms or 6 carbon atoms, i.e., is cyclopropyl, cyclobutyl, cyclopentyl or cyclohepty, although the present definition also covers the occurrence of the term “cycloalkyl” where no numerical range is designated. The term includes fused, non-fused, bridged and spiro radicals. A fused cycloalkyl may contain from two to four fused rings where the ring of attachment is a cycloalkyl ring, and the other individual rings may be alicyclic, heterocyclic, aromatic, heteroaromatic or any combination thereof. Examples include, but are not limited to cyclopropyl, cyclopentyl, cyclohexyl, decalinyl, and bicyclo[2.2.1]heptyl and adamantyl ring systems. Illustrative examples include, but are not limited to the following moieties:

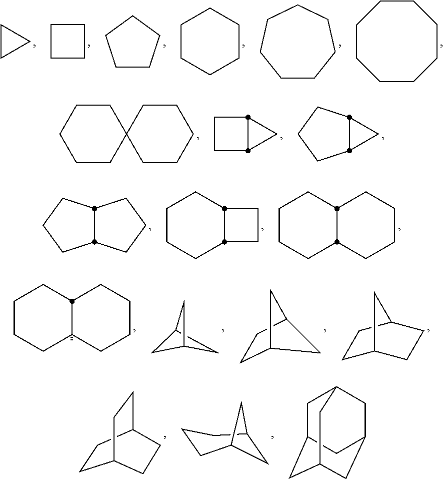

and the like.

In one embodiment, the “cycloalkyl” is substituted. Unless otherwise indicated, the “cycloalkyl” is unsubstituted.

The terms “non-aromatic heterocyclyl” and “heteroalicyclyl” as used herein, alone or in combination, refer to a saturated, partially unsaturated, or fully unsaturated nonaromatic ring monoradicals containing from three to about twenty ring atoms, where one or more of the ring atoms are an atom other than carbon, independently selected from among oxygen, nitrogen, sulfur, phosphorous, silicon, selenium and tin but are not limited to these atoms. In embodiments in which two or more heteroatoms are present in the ring, the two or more heteroatoms can be the same as each another, or some or all of the two or more heteroatoms can each be different from the others. The terms include fused, non-fused, bridged and spiro radicals. A fused non-aromatic heterocyclic radical may contain from two to four fused rings where the attaching ring is a non-aromatic heterocycle, and the other individual rings may be alicyclic, heterocyclic, aromatic, heteroaromatic or any combination thereof. Fused ring systems may be fused across a single bond or a double bond, as well as across bonds that are carbon-carbon, carbon-hetero atom or hetero atom-hetero atom. The terms also include radicals having from three to about twelve skeletal ring atoms, as well as those having from three to about ten skeletal ring atoms. Attachment of a non-aromatic heterocyclic subunit to its parent molecule can be via a heteroatom or a carbon atom. Likewise, additional substitution can be via a heteroatom or a carbon atom. As a non-limiting example, an imidazolidine non-aromatic heterocycle may be attached to a parent molecule via either of its N atoms (imidazolidin-1-yl or imidazolidin-3-yl) or any of its carbon atoms (imidazolidin-2-yl, imidazolidin-4-yl or imidazolidin-5-yl). In certain embodiments, non-aromatic heterocycles contain one or more carbonyl or thiocarbonyl groups such as, for example, oxo- and thio-containing groups. Examples include, but are not limited to pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Illustrative examples of heterocycloalkyl groups, also referred to as non-aromatic heterocycles, include:

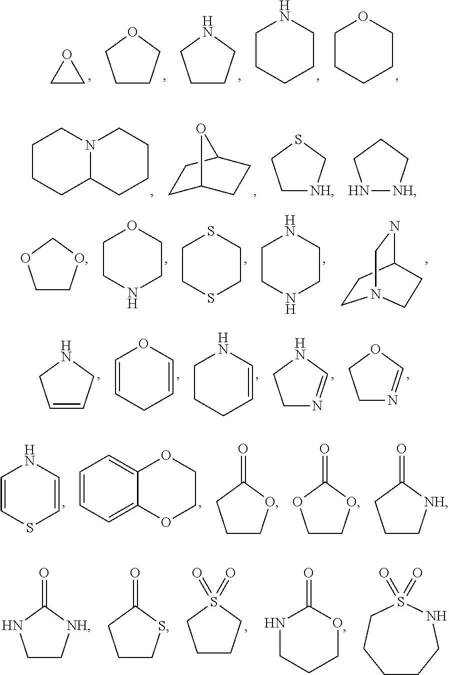

and the like.

The terms also include all ring forms of the carbohydrates, including but not limited to the monosaccharides, the disaccharides and the oligosaccharides. In one embodiment, the “non-aromatic heterocyclyl” or “heteroalicyclyl” is substituted. Unless otherwise indicated, the “non-aromatic heterocyclyl” or “heteroalicyclyl” is unsubstituted.

The term “aryl” as used herein, alone or in combination, refers to an aromatic hydrocarbon radical of six to about twenty ring carbon atoms, and includes fused and non-fused aryl rings. A fused aryl ring radical contains from two to four fused rings where the ring of attachment is an aryl ring, and the other individual rings may be alicyclic, heterocyclic, aromatic, heteroaromatic or any combination thereof. Further, the term aryl includes fused and non-fused rings containing from six to about twelve ring carbon atoms, as well as those containing from six to about ten ring carbon atoms. A non-limiting example of a single ring aryl group includes phenyl; a fused ring aryl group includes naphthyl, phenanthrenyl, anthracenyl, azulenyl; and a non-fused bi-aryl group includes biphenyl. In one embodiment, the “aryl” is substituted. Unless otherwise indicated, the “aryl” is unsubstituted.

The term “heteroaryl” as used herein, alone or in combination, refers to an aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazinyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl and the like, and their oxides, such as for example pyridyl-N-oxide. Illustrative examples of heteroaryl groups include the following moieties:

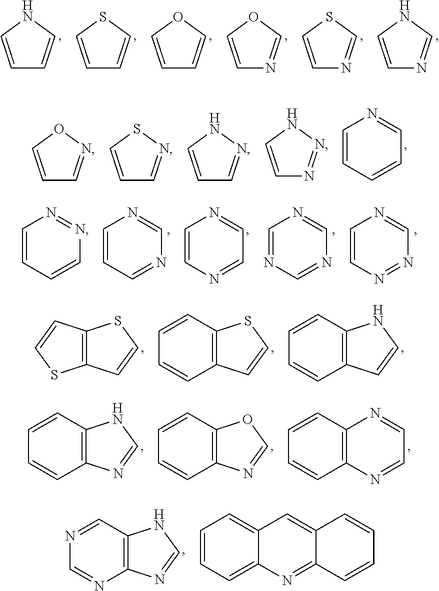

and the like.

In one embodiment, the “heteroaryl” is substituted. Unless otherwise indicated, the “heteroaryl” is unsubstituted.

The term “heterocyclyl” as used herein, alone or in combination, refers collectively to heteroalicyclyl and heteroaryl groups. Herein, whenever the number of carbon atoms in a heterocycle is indicated (e.g., C1-C6 heterocycle), at least one non-carbon atom (the heteroatom) must be present in the ring. Designations such as “C1-C6 heterocycle” refer only to the number of carbon atoms in the ring and do not refer to the total number of atoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). For heterocycles having two or more heteroatoms, those two or more heteroatoms can be the same or different from one another. Non-aromatic heterocyclic groups include groups having only three atoms in the ring, while aromatic heterocyclic groups must have at least five atoms in the ring. Bonding (i.e. attachment to a parent molecule or further substitution) to a heterocycle can be via a heteroatom or a carbon atom. In one embodiment, the “heterocyclyl” is substituted. Unless otherwise indicated, the “heterocycyl” is unsubstituted.

The terms “halogen”, “halo” or “halide” as used herein, alone or in combination refer to fluoro, chloro, bromo and/or iodo.

The term “amino” as used herein, alone or in combination, refers to the monoradical —NH2.

The term “alkylamino” as used herein, alone or in combination, refers to the monoradical —NH(alkyl) where alkyl is as defined herein.

The term “dialkylamino” as used herein, alone or in combination, refers to the monoradical —N(alkyl)(alkyl) where each alkyl may be identical or non-identical and is as defined herein.

The term “diamino alkyl” as used herein, alone or in combination, refers to an alkyl group containing two amine groups, wherein said amine groups may be substituents on the alkyl group which may be amino, alkylamino, or dialkylamino groups, or wherein one or both of said amine groups may form part of an alkyl chain to form -alkylene-N(H or alkyl)-alkylene-N(H or alkyl or alkylene-)(H or alkyl or alkylene-).

The term “hydroxy” as used herein, alone or in combination, refers to the monoradical —OH.

The term “cyano” as used herein, alone or in combination, refers to the monoradical —CN.

The term “cyanomethyl” as used herein, alone or in combination, refers to the monoradical —CH2CN.

The term “nitro” as used herein, alone or in combination, refers to the monoradical —NO2.

The term “oxy” as used herein, alone or in combination, refers to the diradical —O—.

The term “oxo” as used herein, alone or in combination, refers to the diradical ═O.

The term “carbonyl” as used herein, alone or in combination, refers to the diradical —C(═O)—, which may also be written as —C(O)—.

The terms “carboxy” or “carboxyl” as used herein, alone or in combination, refer to the moiety —C(O)OH, which may also be written as —COOH.

The term “alkoxy” as used herein, alone or in combination, refers to an alkyl ether radical, —O-alkyl, including the groups —O-aliphatic and —O-carbocyclyl, wherein the alkyl, aliphatic and carbocyclyl groups may be optionally substituted, and wherein the terms alkyl, aliphatic and carbocyclyl are as defined herein. Non-limiting examples of alkoxy radicals include methoxy, ethoxy, n-propoxy, isopropoxy, n-butoxy, iso-butoxy, sec-butoxy, tert-butoxy and the like.

The term “sulfinyl” as used herein, alone or in combination, refers to the diradical —S(═O)—.

The term “sulfonyl” as used herein, alone or in combination, refers to the diradical —S(═O)₂—.

The terms “sulfonamide”, “sulfonamido” and “sulfonamidyl” as used herein, alone or in combination, refer to the diradical groups —S(═O)₂—NH— and —NH—S(═O)₂—.

The terms “sulfamide”, “sulfamido” and “sulfamidyl” as used herein, alone or in combination, refer to the diradical group —NH—S(═O)₂—NH—.

The term “reactant,” as used herein, refers to a nucleophile or electrophile used to create covalent linkages.

It is to be understood that in instances where two or more radicals are used in succession to define a substituent attached to a structure, the first named radical is considered to be terminal and the last named radical is considered to be attached to the structure in question. Thus, for example, the radical arylalkyl is attached to the structure in question by the alkyl group.

### Certain Pharmaceutical Terminology

The term “MEK inhibitor” as used herein refers to a compound that exhibits an IC50 with respect to MEK activity, of no more than about 100 μM or not more than about 50 μM, as measured in the Mek1 kinase assay described generally herein. “IC50” is that concentration of inhibitor which reduces the activity of an enzyme (e.g., MEK) to half-maximal level. Compounds useful in certain of the combinations and methods described herein preferably exhibit an IC50 with respect to MEK of no more than about 10 μM, more preferably, no more than about 5 μM, even more preferably not more than about 1 μM, and most preferably, not more than about 200 nM, as measured in the Mek1 kinase assay described herein.

The term “Raf inhibitor” or “Raf kinase inhibitor” as used herein refers to a compound that exhibits an IC50 with respect to Raf activity, of no more than about 100 μM or not more than about 50 μM, as measured in the Raf kinase assay described generally herein. “IC50” is that concentration of inhibitor which reduces the activity of an enzyme (e.g., Raf) to half-maximal level. Compounds useful in the certain of the combinations and method described herein preferably exhibit an IC50 with respect to Raf of no more than about 10 μM, more preferably, no more than about 5 μM, even more preferably not more than about 1 μM, and most preferably, not more than about 200 nM, as measured in the Raf kinase assay described generally herein.

The term “subject”, “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

In some embodiments, significance may be determined statistically—in which case two measured parameters may be referred to as statistically significant. In some embodiments, statistical significance may be quantified in terms of a stated confidence interval (CI), e.g. greater than 90%, greater than 95%, greater than 98%, etc. In some embodiments, statistical significance may be quantified in terms of a p value, e.g. less than 0.5, less than 0.1, less than 0.05, etc. The person skilled in the art will recognize these expressions of significance and will know how to apply them appropriately to the specific parameters that are being compared.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The terms “effective amount”, “therapeutically effective amount” or “pharmaceutically effective amount” as used herein, refer to an amount of at least one agent or compound being administered that is sufficient to treat or prevent the particular disease or condition. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In preferred embodiments, the compounds and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, and/or excipients.

The term “carrier” as used herein, refers to relatively nontoxic chemical compounds or agents that facilitate the incorporation of a compound into cells or tissues.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme inhibitor, or a hormone modulator, which diminishes, or prevents the action of another molecule or the activity of a receptor site.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “pharmaceutically acceptable derivative or prodrug” as used herein, refers to any pharmaceutically acceptable salt, ester, salt of an ester or other derivative of a compound of formula I, which, upon administration to a recipient, is capable of providing, either directly or indirectly, a compound of this invention or a pharmaceutically active metabolite or residue thereof. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system).

The term “pharmaceutically acceptable salt” as used herein, includes salts that retain the biological effectiveness of the free acids and bases of the specified compound and that are not biologically or otherwise undesirable. Compounds described may possess acidic or basic groups and therefore may react with any of a number of inorganic or organic bases, and inorganic and organic acids, to form a pharmaceutically acceptable salt. Examples of pharmaceutically acceptable salts include those salts prepared by reaction of the compounds described herein with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorides, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, oxalates, palmoate, pectinate, persulfate, phenylacetates, phenylpropionates, 3-phenylpropionate, phosphate, picrate, pivalate, propionate, pyrosulfate, pyrophosphate, propiolate, propionates, phthalate, phenylbutyrate, propanesulfonate, pyrophosphates, salicylate, succinate, sulfate, sulfite, succinate, suberate, sebacate, sulfonate, tartrate, thiocyanate, tosylate undeconate and xylenesulfonate. Other acids, such as oxalic, while not in themselves pharmaceutically acceptable, may be employed in the preparation of salts useful as intermediates in obtaining the compounds of the invention and their pharmaceutically acceptable acid addition salts. (See for example Berge et al., J. Pharm. Sci. 1977, 66, 1-19.) Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N+(C1-4 alkyl)₄, and the like. Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they may contain. Water or oil-soluble or dispersible products may be obtained by such quaternization. See, for example, Berge et al., supra. These salts can be prepared in situ during the final isolation and purification of the compounds of the invention, or by separately reacting a purified compound in its free base form with a suitable organic or inorganic acid, and isolating the salt thus formed.

The terms “pharmaceutical combination”, “administering an additional therapy”, “administering an additional therapeutic agent” and the like, as used herein, refer to a pharmaceutical therapy resulting from the mixing or combining of more than one active ingredient and includes both fixed and non-fixed combinations of the active ingredients. The term “fixed combination” means that at least one of the compounds described herein, and at least one co-agent, are both administered to a patient simultaneously in the form of a single entity or dosage. The term “non-fixed combination” means that at least one of the compounds described herein, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

The terms “co-administration”, “administered in combination with” and their grammatical equivalents or the like, as used herein, are meant to encompass administration of the selected therapeutic agents to a single patient, and are intended to include treatment regimens in which the agents are administered by the same or different route of administration or at the same or different times. In some embodiments the compounds described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the compounds of the invention and the other agent(s) are administered in a single composition. In some embodiments, compounds of the invention and the other agent(s) are admixed in the composition.

### MEK Protein Kinase Inhibitors

In various embodiments, provided are pharmaceutical combinations comprising a synergistic and therapeutically effective amount of a MEK protein kinase inhibitor and Raf protein kinase inhibitor. In some embodiments, provided are methods of treating cancer comprising the administration of a synergistic and therapeutically effective amount of a pharmaceutical combination, further comprising a MEK protein kinase inhibitor and Raf protein kinase inhibitor.

In further or additional embodiments, provided are pharmaceutical combinations and methods of treating cancer comprising a MEK protein kinase inhibitor. In some embodiments, the MEK protein kinase inhibitor is CI-1040 (PD184352) (Calbiochem), which has the chemical name 2-(2-chloro-4-iodo-phenylamino)-N-cyclopropylmethoxy-3,4-difluoro-benzamide and the following structure:

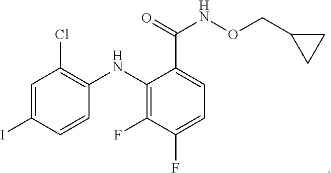 [SMILES: O=C(NOCC1CC1)c1ccc(F)c(F)c1Nc1ccc(I)cc1Cl]

In further embodiments, the MEK protein kinase inhibitor is PD-98059 (Biaffin GmbH & Co. KG; Germany), which has the chemical name 2′-Amino-3′-methoxyflavone and the following chemical structure:

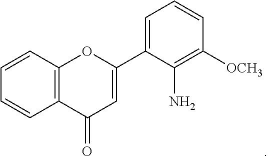 [SMILES: COc1cccc(-c2cc(=O)c3ccccc3o2)c1N]

In some embodiments, the MEK protein kinase inhibitor is UO126 (Biaffin GmbH & Co. KG; Germany), which has the chemical name 1,4-Diamino-2,3-dicyano-1,4-bis(2-aminophenylthio)-butadiene and the following chemical structure:

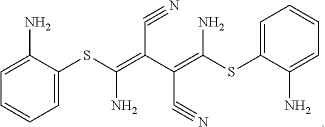 [SMILES: N#CC(=C(/N)Sc1ccccc1N)/C(C#N)=C(\N)Sc1ccccc1N]

In further embodiments, the MEK protein kinase inhibitor is SL 327 (Biaffin GmbH & Co. KG; Germany), which has the chemical name α-[Amino[(4-aminophenyl)thio]methylene]-2-(trifluoromethyl)benzeneacetonitrile and the following chemical structure:

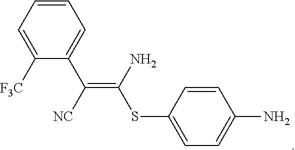 [SMILES: N#C/C(=C(/N)Sc1ccc(N)cc1)c1ccccc1C(F)(F)F]

In other embodiments, the MEK protein kinase inhibitor is the phytochecmial quercetin, which has the chemical name 2-(3,4-dihydroxyphenyl)-3,5,7-trihydroxy-4H-chromen-4-one and the following chemical structure:

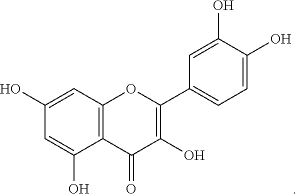 [SMILES: O=c1c(O)c(-c2ccc(O)c(O)c2)oc2cc(O)cc(O)c12]

In additional embodiments, the MEK protein kinase inhibitor is PD-184161, which has the chemical name 2-(2-Chloro-4-iodophenylamino)-N-cyclopropylmethoxy-3,4-difluoro-5-bromobenzamide.

In further or additional embodiments, the MEK protein kinase inhibitor is GSK1120212. In some embodiments, the MEK protein kinase inhibitor is PD-0325901. In further embodiments, the MEK protein kinase inhibitor is PD 0318088. In other embodiments, the MEK protein kinase inhibitor is PD-184386. In some embodiments, the MEK protein kinase inhibitor is PD-170611. In additional embodiments, the MEK protein kinase inhibitor is PD-177168. In further embodiments, the MEK protein kinase inhibitor is PD-184352. In further embodiments, the MEK protein kinase inhibitor is PD-171984. In other embodiments, the MEK protein kinase inhibitor is ARRY-438162. In some embodiments, the MEK protein kinase inhibitor is AZD6244/ARRY-886. In additional embodiments, the MEK protein kinase inhibitor is AZD 8330. In further embodiments, the MEK protein kinase inhibitor is XL518. In one embodiment, the MEK protein kinase inhibitor is UO125 (Calbiochem), which has the chemical name.

In further or additional embodiments where the MEK protein kinase inhibitor is a compound is selected from the group of compounds consisting of:

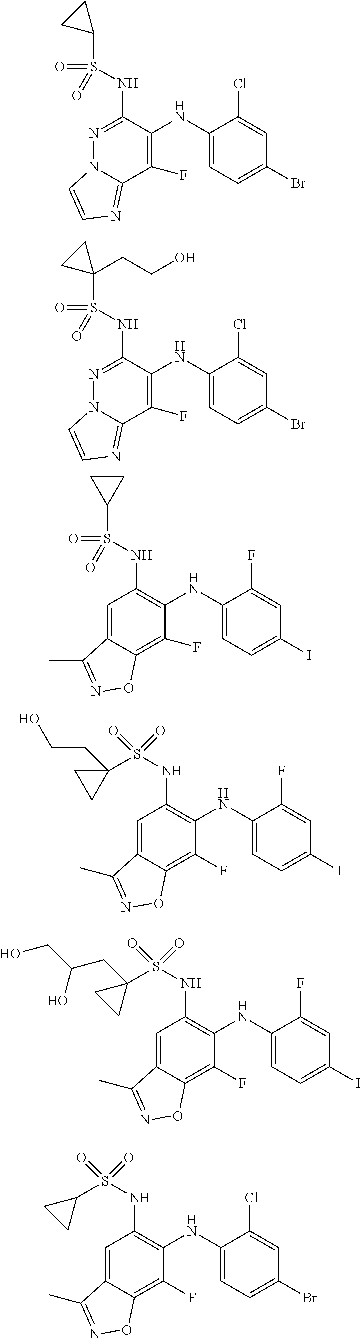

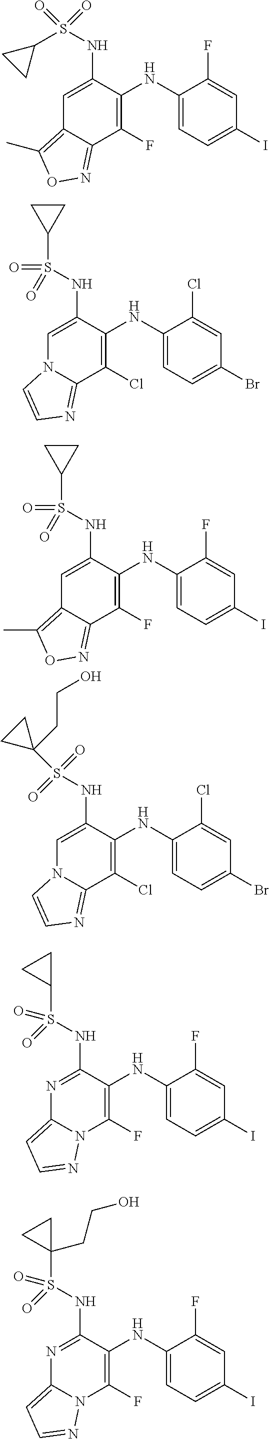

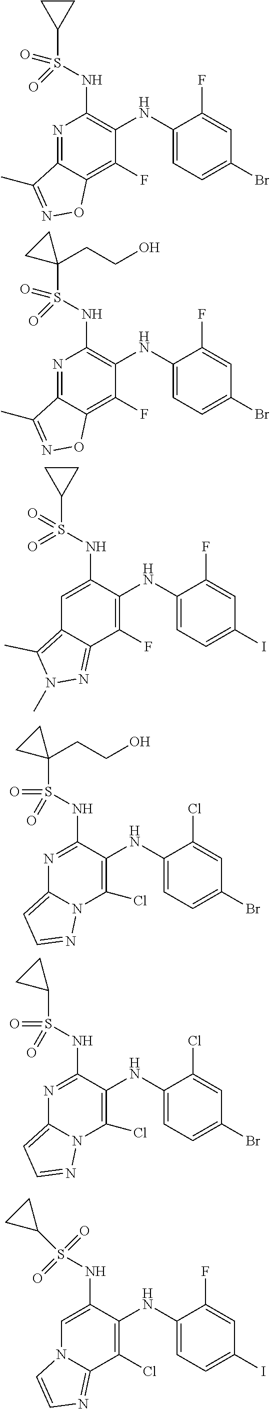

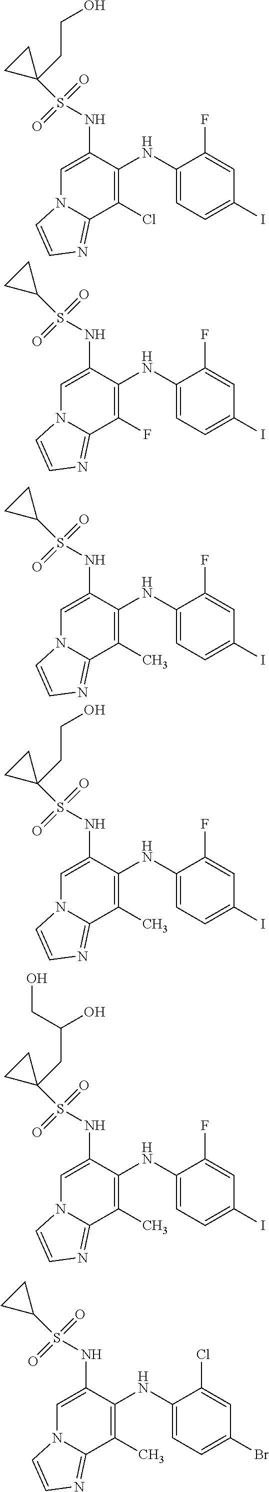

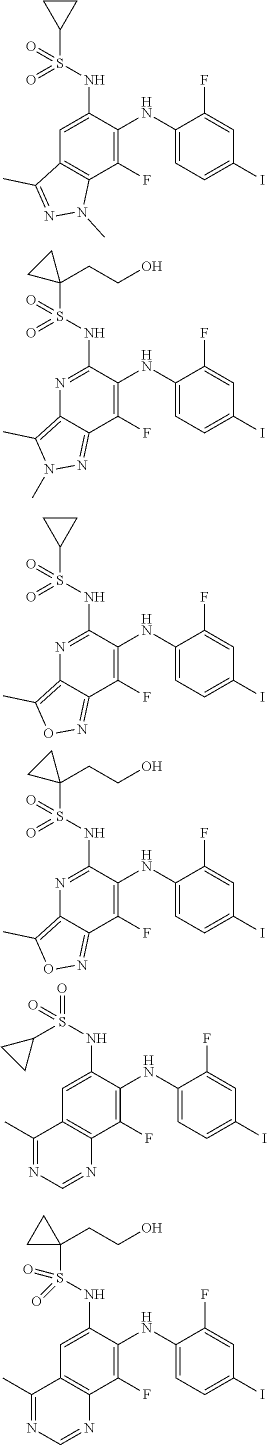

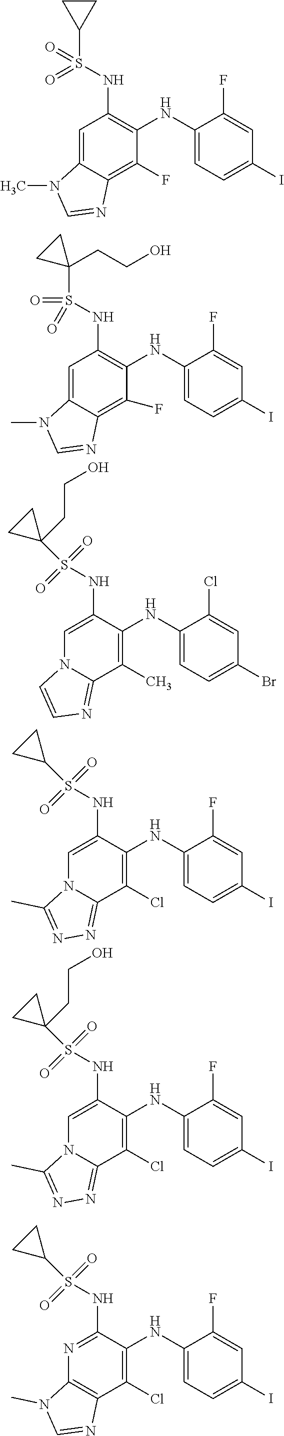

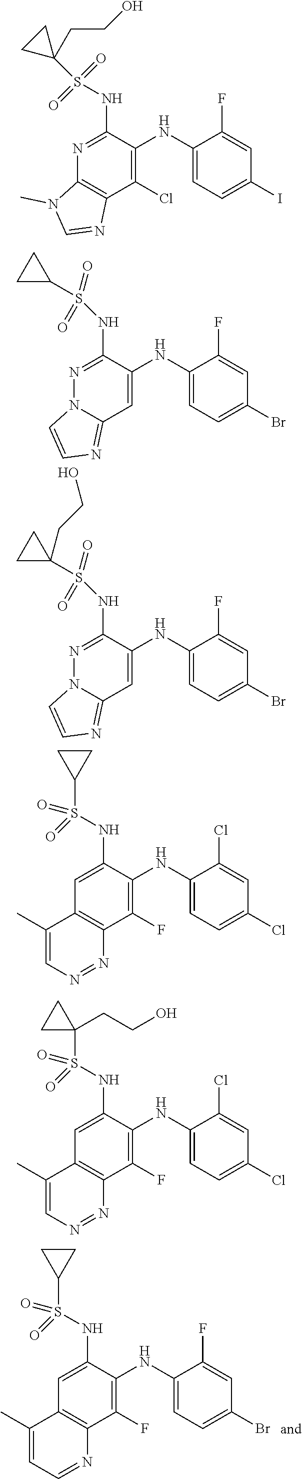

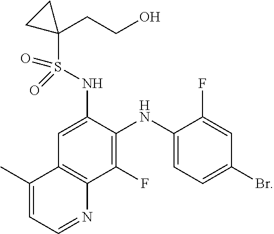 [SMILES: Cc1ccnc2c(F)c(Nc3ccc(Br)cc3F)c(NS(=O)(=O)C3(CCO)CC3)cc12]

### Exemplary Effects of the Pharmaceutical Combination of MEK Inhibitors and Sorafenib or Regorafenib Pharmaceutical Compositions of MEK Inhibitors and Sorafenib or Regorafenib

In further aspects, the present invention is directed to a pharmaceutical composition comprising a MEK protein kinase inhibitor and sorafenib or Regorafenib, wherein the MEK protein kinase inhibitor is a compound of formula A. In some embodiments, the pharmaceutical compositions further comprise a pharmaceutically acceptable carrier. Such compositions may contain adjuvants, excipients, and preservatives, agents for delaying absorption, fillers, binders, adsorbents, buffers, disintegrating agents, solubilizing agents, other carriers, and other inert ingredients. Methods of formulation of such compositions are well-known in the art.

In some embodiments, the pharmaceutical composition is in a form suitable for oral administration. In further or additional embodiments, the pharmaceutical composition is in the form of a tablet, capsule, pill, powder, sustained release formulation, solution, suspension, for parenteral injection as a sterile solution, suspension or emulsion, for topical administration as an ointment or cream or for rectal administration as a suppository. In further or additional embodiments, the pharmaceutical composition is in unit dosage forms suitable for single administration of precise dosages.

In further or additional embodiments, the amount of the MEK protein kinase inhibitor is in the range of about 0.001 to about 1000 mg/kg body weight/day. In further or additional embodiments the amount of MEK protein kinase inhibitor is in the range of about 0.5 to about 50 mg/kg/day. In further or additional embodiments the amount of MEK protein kinase inhibitor is about 0.001 to about 7 g/day. In further or additional embodiments the amount of MEK protein kinase inhibitor is about 0.002 to about 6 g/day. In further or additional embodiments the amount of MEK protein kinase inhibitor is about 0.005 to about 5 g/day. In further or additional embodiments the amount of MEK protein kinase inhibitor is about 0.01 to about 5 g/day. In further or additional embodiments the amount of MEK protein kinase inhibitor is about 0.02 to about 5 g/day. In further or additional embodiments the amount of MEK protein kinase inhibitor is about 0.05 to about 2.5 g/day. In further or additional embodiments the amount of MEK protein kinase inhibitor is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the MEK protein kinase inhibitor and sorafenib or Regorafenib in combination is administered in a single dose, once daily.

In some embodiments the MEK inhibitor and Raf inhibitor are administered on different timing regimens.

In some embodiments, the pharmaceutical composition is for administration to a mammal. In further or additional embodiments, the mammal is human. In further or additional embodiments, the pharmaceutical composition further comprises a pharmaceutical carrier, excipient and/or adjuvant.

In further or additional embodiments, the pharmaceutical composition further comprises at least one additional therapeutic agent. In further or additional embodiments, the therapeutic agent is selected from the group of cytotoxic agents, anti-angiogenesis agents and anti-neoplastic agents. In further or additional embodiments, the anti-neoplastic agent is selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors. In further or additional embodiments, the therapeutic agent is taxol, bortezomib or both. In further or additional embodiments, the pharmaceutical composition is administered in combination with an additional therapy. In further or additional embodiments, the additional therapy is radiation therapy, chemotherapy, surgery or any combination thereof.

### Tumor Size/Tumor Load/Tumor Burden

In other aspects, the present invention is directed to a method of reducing the size of a tumor, inhibiting tumor size increase, reducing tumor proliferation or preventing tumor proliferation in an individual, comprising administering to said individual an effective amount of a MEK protein kinase inhibitor and/or Sorafenib or Regorafenib. In some embodiments, combination is administered as a component of a composition that further comprises a pharmaceutically acceptable carrier or vehicle. In some embodiments, the size of a tumor is reduced. In further or additional embodiments, the size of a tumor is reduced by at least 1%. In further or additional embodiments, the size of a tumor is reduced by at least 2%. In further or additional embodiments, the size of a tumor is reduced by at least 3%. In further or additional embodiments, the size of a tumor is reduced by at least 4%. In further or additional embodiments, the size of a tumor is reduced by at least 5%. In further or additional embodiments, the size of a tumor is reduced by at least 10%. In further or additional embodiments, the size of a tumor is reduced by at least 20%. In further or additional embodiments, the size of a tumor is reduced by at least 25%. In further or additional embodiments, the size of a tumor is reduced by at least 30%. In further or additional embodiments, the size of a tumor is reduced by at least 40%. In further or additional embodiments, the size of a tumor is reduced by at least 50%. In further or additional embodiments, the size of a tumor is reduced by at least 60%. In further or additional embodiments, the size of a tumor is reduced by at least 70%. In further or additional embodiments, the size of a tumor is reduced by at least 75%. In further or additional embodiments, the size of a tumor is reduced by at least 80%. In further or additional embodiments, the size of a tumor is reduced by at least 85%. In further or additional embodiments, the size of a tumor is reduced by at least 90%. In further or additional embodiments, the size of a tumor is reduced by at least 95%. In further or additional embodiments, the tumor is eradicated. In some embodiments, the size of a tumor does not increase.

In some embodiments, tumor proliferation is reduced. In some embodiments, tumor proliferation is reduced by at least 1%. In some embodiments, tumor proliferation is reduced by at least 2%. In some embodiments, tumor proliferation is reduced by at least 3%. In some embodiments, tumor proliferation is reduced by at least 4%. In some embodiments, tumor proliferation is reduced by at least 5%. In some embodiments, tumor proliferation is reduced by at least 10%. In some embodiments, tumor proliferation is reduced by at least 20%. In some embodiments, tumor proliferation is reduced by at least 25%. In some embodiments, tumor proliferation is reduced by at least 30%. In some embodiments, tumor proliferation is reduced by at least 40%. In some embodiments, tumor proliferation is reduced by at least 50%. In some embodiments, tumor proliferation is reduced by at least 60%. In some embodiments, tumor proliferation is reduced by at least 70%. In some embodiments, tumor proliferation is reduced by at least 75%. In some embodiments, tumor proliferation is reduced by at least 75%. In some embodiments, tumor proliferation is reduced by at least 80%. In some embodiments, tumor proliferation is reduced by at least 90%. In some embodiments, tumor proliferation is reduced by at least 95%. In some embodiments, tumor proliferation is prevented.

In some embodiments, the combination is administered in combination with an additional therapy. In further or additional embodiments, the additional therapy is radiation therapy, chemotherapy, surgery or any combination thereof. In further or additional embodiments, the combination is administered in combination with at least one therapeutic agent. In further or additional embodiments, the therapeutic agent is selected from the group of cytotoxic agents, anti-angiogenesis agents and anti-neoplastic agents. In further or additional embodiments, the anti-neoplastic agent is selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors. In further or additional embodiments, the therapeutic agent is selected from taxol, bortezomib or both.

In some embodiments, the composition comprising a MEK protein kinase inhibitor and sorafenib or Regorafenib is administered orally, intraduodenally, parenterally (including intravenous, subcutaneous, intramuscular, intravascular or by infusion), topically or rectally. In further or additional embodiments the amount of compound of formula A is in the range of about 0.001 to about 1000 mg/kg body weight/day. In further or additional embodiments the amount of compound of formula A, is in the range of about 0.5 to about 50 mg/kg/day. In further or additional embodiments the amount of compound of formula A is about 0.001 to about 7 g/day. In further or additional embodiments the amount of compound of formula A is about 0.01 to about 7 g/day. In further or additional embodiments the amount of compound of formula A is about 0.02 to about 5 g/day. In further or additional embodiments the amount of compound of formula S is about 0.05 to about 2.5 g/day. In further or additional embodiments the amount of compound of formula A is about 0.1 to about 1 g/day. In further or additional embodiments, dosage levels below the lower limit of the aforesaid range may be more than adequate. In further or additional embodiments, dosage levels above the upper limit of the aforesaid range may be required.

### Modes of Administration

Described herein are MEK protein kinase inhibitor and sorafenib or Regorafenib combinations. Also described are pharmaceutical compositions comprising a MEK protein kinase and Sorafenib or Regorafenib. The difference between a “combination” and a “composition” as used herein is that the MEK inhibitor and Raf inhibitor may be in different dosage forms in the “combination,” but are in the same dosage form in the “composition.” The compounds and compositions described herein may be administered either alone or in combination with pharmaceutically acceptable carriers, excipients or diluents, in a pharmaceutical composition, according to standard pharmaceutical practice.

Administration of the compounds and compositions described herein can be effected by any method that enables delivery of the compounds to the site of action. These methods include oral routes, intraduodenal routes, parenteral injection (including intravenous, subcutaneous, intraperitoneal, intramuscular, intravascular or infusion), topical, and rectal administration. For example, compounds described herein can be administered locally to the area in need of treatment. This may be achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue. Those of ordinary skill in the art are familiar with formulation and administration techniques that can be employed with the compounds and methods of the invention, e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, solvate, polymorph, ester, amide, tautomer, prodrug, hydrate, or derivative thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which are useful for oral administration include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules or tablets can contain the active ingredient; in admixture with a filler such as microcrystalline cellulose, silicified microcrystalline cellulose, pregelatinized starch, lactose, dicalcium phosphate, or compressible sugar; a binder such as hypromellose, povidone or starch paste; a disintegrant such as croscarmellose sodium, crospovidone or sodium starch glycolate; a surfactant such as sodium lauryl sulfate and/or lubricants and processing aides such as talc, magnesium stearate, stearic acid or colloidal silicon dioxide and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions are useful, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses

### Formulations

Pharmaceutical preparations may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical preparations may also be formulated in rectal compositions such as suppositories or retention enemas, e.g., containing conventional suppository bases such as cocoa butter, polyethylene glycol, or other glycerides.

Pharmaceutical preparations may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical preparations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w or may comprise less than 5% w/w, or from 0.1% to 1% w/w of the formulation.

Pharmaceutical preparations for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

The compounds or compositions described herein can be delivered in a vesicle, e.g., a liposome (see, for example, Langer, Science 1990, 249, 1527-1533; Treat et al., Liposomes in the Therapy of Infectious Disease and Cancer, Lopez-Bernstein and Fidler, Ed., Liss, N.Y., pp. 353-365, 1989). The compounds and pharmaceutical compositions described herein can also be delivered in a controlled release system. In one embodiment, a pump may be used (see, Sefton, 1987, CRC Crit. Ref. Biomed. Eng. 14:201; Buchwald et al. Surgery, 1980 88, 507; Saudek et al. N. Engl. J. Med. 1989, 321, (574). Additionally, a controlled release system can be placed in proximity of the therapeutic target. (See, Goodson, Medical Applications of Controlled Release, 1984, Vol. 2, pp. 115-138). The pharmaceutical compositions described herein can also contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents; fillers such as microcrystalline cellulose, silicified microcrystalline cellulose, pregelatinized starch, lactose, dicalcium phosphate, or compressible sugar; binders such as hypromellose, povidone or starch paste; disintegrants such as croscarmellose sodium, crospovidone or sodium starch glycolate; a surfactant such as sodium lauryl sulfate and/or lubricants and processing aides such as talc, sodium croscarmellose, corn starch, or alginic acid; binding agents, for example starch, gelatin, polyvinyl-pyrrolidone or acacia, and lubricating agents, for example magnesium stearate, stearic acid or colloidal silicon dioxide and, optionally, talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil. The capsule and tablet dosage forms may be prepared by various processing techniques including dry blending and wet granulation techniques. In the dry blending method of manufacture the drug substance may be incorporated into the dosage form by dry blending with the excipients followed by encapsulation into a capsule shell or compression into a tablet form. The dry blending operation may be approached in a stepwise manner and include screening steps between the blending steps to facilitate formation of a uniform blend. In the wet granulation method of manufacture the drug substance may be added to the dry excipients and mixed prior to the addition of the binder solution or the drug substance may be dissolved and added as a solution as part of granulation. In the wet granulation technique the surfactant, if used, may be added to the dry excipients or added to the binder solution and incorporated in a solution form. Capsule dosage forms may also be manufactured by dissolving the drug substance in a material that can be filled into and is compatible with hard gelatin capsule shells that can be subsequently banded and sealed. Capsule and tablet dosage forms may also be produced by dissolving the drug substance in a material such a molten form of a high molecular weight polyethylene glycol and cooling to a solid form, milling and incorporating this material into conventional capsule and tablet manufacturing processes.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solution. Among the acceptable vehicles and solvents that may be employed are water, Ringer's solution and isotonic sodium chloride solution. The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion. The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump. The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Pharmaceutical compositions may also be administered in the form of suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing a compound or composition of the invention are useful for topical administration. As used herein, topical application can include mouth washes and gargles.

Pharmaceutical compositions may be administered in intranasal form via topical use of suitable intranasal vehicles and delivery devices, or via transdermal routes, using those forms of transdermal skin patches well known to those of ordinary skill in the art.

The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation. Methods of preparing various pharmaceutical compositions with a specific amount of active compound are known, or will be apparent, to those skilled in this art. To be administered in the form of transdermal delivery, the dosage form will, of course, be continuous rather than intermittent throughout the dosage regimen.

### Doses

### Dosage Amounts of MEK Inhibitors

The amount of pharmaceutical combination of MEK protein kinase inhibitor and sorafenib or Regorafenib administered will firstly be dependent on the mammal being treated. In the instances where pharmaceutical compositions are administered to a human subject, the daily dosage will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, the disposition of the composition, rate of excretion, drug combination, and the discretion of the prescribing physician. Also, the route of administration may vary depending on the condition and its severity. The pharmaceutical composition may be in unit dosage form. In such form, the preparation is subdivided into unit doses containing appropriate quantities of the active component, e.g., an effective amount to achieve the desired purpose. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. The amount and frequency of administration of the compounds described herein, and if applicable other therapeutic agents and/or therapies, will be regulated according to the judgment of the attending clinician (physician) considering such factors as described above. Thus the amount of pharmaceutical composition to be administered may vary widely. Administration may occur in an amount of between about 0.001 mg/kg of body weight to about 100 mg/kg of body weight per day (administered in single or divided doses), or at least about 0.1 mg/kg of body weight per day. A particular therapeutic dosage can include, e.g., from about 0.01 mg to about 7000 mg of compound, or, e.g., from about 0.05 mg to about 2500 mg. The quantity of active compound in a unit dose of preparation may be varied or adjusted from about 0.1 mg to 1000 mg, from about 1 mg to 300 mg, or 10 mg to 200 mg, according to the particular application. In some instances, dosage levels below the lower limit of the aforesaid range may be more than adequate, while in other cases still larger doses may be employed without causing any harmful side effect, e.g. by dividing such larger doses into several small doses for administration throughout the day. The amount administered will vary depending on the particular IC50 value of the compound used. In combinational applications in which the compound is not the sole therapy, it may be possible to administer lesser amounts of compound and still have therapeutic or prophylactic effect.

In another aspect, provided herein are pharmaceutical combinations and methods of treating cancer comprising a therapeutically effective amount of a MEK protein kinase inhibitor and sorafenib or Regorafenib, wherein the combination allows for particular dosing.

In some embodiments of the combinations and methods provided herein, the molar ratio of the MEK protein kinase inhibitor to sorafenib or Regorafenib administered to a patient is about 100:1 to about 2.5:1. In other embodiments, the molar ratio of the MEK protein kinase inhibitor to sorafenib or Regorafenib administered to a patient is about 50:1 to about 5:1. In other embodiments, the molar ratio of the MEK protein kinase inhibitor to the Sorafenib or Regorafenib administered to a patient is about 45:1 to about 10:1. In other embodiments, the molar ratio of the MEK protein kinase inhibitor to sorafenib or Regorafenib administered to a patient is about 40:1 to about 20:1. In other embodiments, the molar ratio of the MEK protein kinase inhibitor to sorafenib or Regorafenib administered to a patient is about 30:1.

### Dosage Amounts of Sorafenib

In another aspect, the combinations and methods described herein provide Sorafenib or Regorafenibs. In some embodiments, the sorafenib is present in an amount of about 10 mg to about 1,000 mg. In further or additional embodiments, the sorafenib is present in an amount of about 20 mg to about 900 mg. In further embodiments, the sorafenib is present in an amount of about 20 mg to about 900 mg. In still further embodiments, the sorafenib is present in an amount of about 30 mg to about 850 mg. In certain embodiments, the sorafenib is present in an amount of about 40 mg to about 800 mg. In still further embodiments, the sorafenib is present in an amount of about 50 mg to about 750 mg. In other embodiments, the sorafenib is present in an amount of about 75 mg to about 700 mg, about 100 mg to about 650 mg, about 150 mg to about 600 mg, about 200 mg to about 500 mg, about 300 mg to about 400 mg.

In further or additional embodiments of the pharmaceutical combinations and methods described herein, the Sorafenib or Regorafenib is sorafenib and is present in an amount of about 10 mg, about 20 mg, about 30 mg, about 40 mg, about 50 mg, about 60 mg, about 70 mg, about 80 mg, about 90 mg, about 100 mg, about 125 mg, about 150 mg, about 200 mg, about 250 mg, about 300 mg, about 350 mg, about 400 mg, about 450 mg, about 500 mg, about 600 mg, about 700 mg, about 750 mg, about 800 mg, about 850 mg, about 900 mg, about 950 mg, or about 1000 mg.

### Dosage Forms

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules, including lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Ester, Pa., 18th Edition (1990).

### Kits

The present application concerns kits for use with the compounds described herein. In some embodiments, the invention provides a kit including an MEK protein kinase inhibitor and/or sorafenib or Regorafenib in a dosage form, particularly a dosage form for oral administration. In some embodiments, the kit further includes a MEK protein kinase inhibitor and/or sorafenib or Regorafenib in a dosage form. In specific embodiments, the MEK protein kinase inhibitor and/or sorafenib or Regorafenib are in separate dosage forms. In other embodiments, the MEK protein kinase inhibitor and/or sorafenib or Regorafenib are in the same dosage form. In some embodiments, the kit includes one or more doses of a MEK protein kinase inhibitor and/or sorafenib or Regorafenib in tablets for oral administration. In other embodiments, however, the dose or doses of MEK protein kinase inhibitor and/or sorafenib or Regorafenib may be present in a variety of dosage forms, such as capsules, caplets, gel caps, powders for suspension, etc. In some embodiments, the kit includes one or more doses of an MEK protein kinase inhibitor and/or sorafenib or Regorafenib for oral administration. In other embodiments, however, the dose or doses of an MEK protein kinase inhibitor and/or sorafenib or Regorafenib may be present in a variety of dosage forms, such as capsules, caplets, gel caps, powders for suspension, etc.

The container means of the kits will generally include at least one vial, test tube, flask, bottle, syringe and/or other container means, into which the at least one polypeptide can be placed, and/or preferably, suitably aliquoted. The kits can include a means for containing at least one fusion protein, detectable moiety, reporter molecule, and/or any other reagent containers in close confinement for commercial sale. Such containers may include injection and/or blow-molded plastic containers in which the desired vials are stored. Kits can also include printed material for use of the materials in the kit.

Packages and kits can additionally include a buffering agent, a preservative and/or a stabilizing agent in a pharmaceutical formulation. Each component of the kit can be enclosed within an individual container and all of the various containers can be within a single package. Invention kits can be designed for cold storage or room temperature storage.

Additionally, the preparations can contain stabilizers (such as bovine serum albumin (BSA)) to increase the shelf-life of the kits. Where the compositions are lyophilized, the kit can contain further preparations of solutions to reconstitute the lyophilized preparations. Acceptable reconstitution solutions are well known in the art and include, for example, pharmaceutically acceptable phosphate buffered saline (PBS).

Additionally, the packages or kits provided herein can further include any of the other moieties provided herein such as, for example, one or more reporter molecules and/or one or more detectable moieties/agents.

Packages and kits can further include one or more components for an assay, such as, for example, an ELISA assay, cytotoxicity assay, ADP-Ribosyltransferase activity assay, etc. Samples to be tested in this application include, for example, blood, plasma, and tissue sections and secretions, urine, lymph, and products thereof. Packages and kits can further include one or more components for collection of a sample (e.g., a syringe, a cup, a swab, etc.).

Packages and kits can further include a label specifying, for example, a product description, mode of administration and/or indication of treatment. Packages provided herein can include any of the compositions as described herein for treatment of any of the indications described herein.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions. Kits, therefore, can additionally include labels or instructions for using the kit components in any method of the invention. A kit can include a compound in a pack, or dispenser together with instructions for administering the compound in a method described herein.

In some embodiments, a kit includes at least three dosage forms, one comprising an MEK protein kinase inhibitor, one comprising Sorafenib or Regorafenib and the other comprising at least a third active pharmaceutical ingredient, other than the MEK protein kinase inhibitor or Sorafenib or Regorafenib. In some embodiments, the third active pharmaceutical ingredient is a second MEK protein kinase inhibitor. In other embodiments, the third active pharmaceutical ingredient is a second Sorafenib or Regorafenib. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. It is considered that the most convenient periods of time for which such kits are designed would be from 1 to 13 weeks, especially 1 week, 2 weeks, 1 month, 3 months, etc. In some specific embodiments, the each dose is physically separated into a compartment, in which each dose is segregated from the others.

In some embodiments, the kit includes at least two dosage forms one comprising a MEK protein kinase inhibitor and one comprising Sorafenib or Regorafenib. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In particular embodiments, the kit may advantageously be a blister pack. Blister packs are known in the art, and generally include a clear side having compartments (blisters or bubbles), which separately hold the various doses, and a backing, such as a paper, foil, paper-foil or other backing, which is easily removed so that each dose may be separately extracted from the blister pack without disturbing the other doses. In some embodiments, the kit may be a blister pack in which each dose of the MEK protein kinase inhibitor, sorafenib or Regorafenib and, optionally, a third active pharmaceutical ingredient are segregated from the other doses in separate blisters or bubbles. In some such embodiments, the blister pack may have perforations, which allow each daily dose to be separated from the others by tearing it away from the rest of the blister pack. The separate dosage forms may be contained within separate blisters. Segregation of the active pharmaceutical ingredients into separate blisters can be advantageous in that it prevents separate dosage forms (e.g., tablet and capsule) from contacting and damaging one another during shipping and handling. Additionally, the separate dosage forms can be accessed and/or labeled for administration to the patient at different times.

In some embodiments, the kit may be a blister pack in which each separate dose the MEK protein kinase inhibitor, Sorafenib or Regorafenib, and, optionally, a third active pharmaceutical ingredient is segregated from the other doses in separate blisters or bubbles. In some such embodiments, the blister pack may have perforations, which allow each daily dose to be separated from the others by tearing it away from the rest of the blister pack. The separate dosage forms may be contained within separate blisters.

In some embodiments, the third active pharmaceutical ingredient may be in the form of a liquid or a reconstitutable powder, which may be separately sealed (e.g., in a vial or ampoule) and then packaged along with a blister pack containing separate dosages of the MEK protein kinase inhibitor and Sorafenib or Regorafenib. In some embodiments, the MEK protein kinase inhibitor is in the form of a liquid or reconstitutable powder that is separately sealed (e.g., in a vial or ampoule) and then packaged along with a blister pack containing separate dosages of the MEK protein kinase inhibitor. These embodiments would be especially useful in a clinical setting where prescribed doses of the MEK protein kinase inhibitor, Sorafenib or Regorafenib, and, optionally, a third active pharmaceutically active agent would be used on a dosing schedule in which the MEK protein kinase inhibitor and Sorafenib or Regorafenib is each administered on certain days, Sorafenib or Regorafenib is administered on the same or different days and the third active pharmaceutical ingredient is administered on the same or different days from either or both of the MEK protein kinase inhibitor and/or Sorafenib or Regorafenib within a weekly, bi-weekly, 2× weekly or other dosing schedule. Such a combination of blister pack containing a MEK protein kinase inhibitor, Sorafenib or Regorafenib and an optional third active pharmaceutical agent could also include instructions for administering each of the MEK protein kinase inhibitor, Sorafenib or Regorafenib, and the optional third active pharmaceutical agent on a dosing schedule adapted to provide the synergistic or sequelae-treating effect of the MEk protein kinase inhibitor and/or the third active pharmaceutical agent.

In other embodiments, the kit may be a container having separate compartments with separate lids adapted to be opened on a particular schedule. For example, a kit may comprise a box (or similar container) having seven compartments, each for a separate day of the week, and each compartment marked to indicate which day of the week it corresponds to. In some specific embodiments, each compartment is further subdivided to permit segregation of one active pharmaceutical ingredient from another. As stated above, such segregation is advantageous in that it prevents damage to the dosage forms and permits dosing at different times and labeling to that effect. Such a container could also include instructions for administering a MEK protein kinase inhibitor, Sorafenib or Regorafenib and the optional third active pharmaceutical ingredient on a dosing schedule adapted to provide the synergistic or sequelae-treating effect of the MEK protein kinase inhibitor and/or the third active pharmaceutical ingredient.

The kits may also include instructions teaching the use of the kit according to the various methods and approaches described herein. Such kits optionally include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. Such information may be based on the results of various studies, for example, studies using experimental animals involving in vivo models and studies based on human clinical trials. In various embodiments, the kits described herein can be provided, marketed and/or promoted to health providers, including physicians, nurses, pharmacists, formulary officials, and the like. Kits may, in some embodiments, be marketed directly to the consumer. In certain embodiments, the packaging material further comprises a container for housing the composition and optionally a label affixed to the container. The kit optionally comprises additional components, such as but not limited to syringes for administration of the composition.

Instructions can include instructions for practicing any of the methods described herein including treatment methods. Instructions can additionally include indications of a satisfactory clinical endpoint or any adverse symptoms that may occur, or additional information required by regulatory agencies such as the Food and Drug Administration for use on a human subject.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM, IC tip and hybrids of these such as magnetic/optical storage media.

In some embodiments, the kit comprises a MEK protein kinase inhibitor that is visibly different from Sorafenib or Regorafenib. In certain embodiments, each of the MEK protein kinase inhibitor dosage form and Sorafenib or Regorafenib dosage form are visibly different from a third pharmaceutical agent dosage form. The visible differences may be for example shape, size, color, state (e.g., liquid/solid), physical markings (e.g., letters, numbers) and the like. In certain embodiments, the kit comprises a MEK protein kinase inhibitor (e.g. compound A or compound B) dosage form that is a first color, Sorafenib or Regorafenib dosage form that is a second color, and an optional third pharmaceutical composition that is a third color. In embodiments wherein the first, second and third colors are different, the different colors of the first, second and third pharmaceutical compositions is used, e.g., to distinguish between the first, second and third pharmaceutical compositions.

In some embodiments, wherein the packaging material further comprises a container for housing the pharmaceutical composition, the kit comprises a MEK protein kinase inhibitor composition that is in a different physical location within the kit from Sorafenib or Regorafenib composition. In further embodiments, the kit comprises a third pharmaceutical agent that is in a separate physical location from either the Mek protein kinase inhibitor composition or Sorafenib or Regorafenib composition. In some embodiments, the different physical locations of MEK protein kinase inhibitor composition and Sorafenib or Regorafenib composition comprise separately sealed individual compartments. In certain embodiments, the kit comprises a MEK protein kinase inhibitor composition that is in a first separately sealed individual compartment and Sorafenib or Regorafenib composition that is in a second separately sealed individual compartment. In embodiments wherein the MEK protein kinase inhibitor and Sorafenib or Regorafenib composition compartments are separate, the different locations are used, e.g., to distinguish between the MEK protein kinase inhibitor composition and Sorafenib or Regorafenib compositions. In further embodiments, a third pharmaceutical composition is in a third physical location within the kit.

The compounds described herein can be utilized for diagnostics and as research reagents. For example, the compounds described herein, either alone or in combination with other compounds, can be used as tools in differential and/or combinatorial analyses to elucidate expression patterns of genes expressed within cells and tissues. As one non-limiting example, expression patterns within cells or tissues treated with one or more compounds are compared to control cells or tissues not treated with compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Besides being useful for human treatment, the compounds and formulations of the present invention are also useful for veterinary treatment of companion animals (eg dogs, cats), exotic animals and farm animals (eg horses), including mammals, rodents, and the like.

Hence, in a sixth aspect, the invention is directed to the use of a compound of formula A as defined herein, for the preparation of a medicament for the treatment of hepatocellular carcinoma in a patient possessing a mutated KRAS, NRAS or HRAS gene.

The examples and preparations provided below further illustrate and exemplify the compounds of the present invention and methods of preparing such compounds. It is to be understood that the scope of the present invention is not limited in any way by the scope of the following examples and preparations.

### EXAMPLES

### Synthesis of Compounds

### Example 1

### N-(3,4-difluoro-2-(2-fluoro-4-iodophenylamino)phenyl)-1-(2,3-dihydroxypropyl)cyclopropane-1-sulfonamide

### Step A: Butyl cyclopropanesulfonate

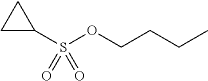

Cyclopropanesulfonyl chloride (5 g, 35 mmol, 1 eq) was dissolved in an excess BuOH (20 ml), the reaction mixture was cooled at −10° C. and pyridine (5.8 mL, 70 mmol, 2 eq) was slowly added dropwise. The mixture was slowly warmed at room temperature and stirred overnight. The solvent was removed under reduced pressure and the resulting white solid was dissolved in CHCl₃. The organic phase was washed with water, brine and dried (MgSO4) and concentrated to give an oil (4.8 g, 24.9 mmol, 71%). ¹H NMR (300 MHz, CDCl₃): δ 4.25 (t, 2H), 2.46 (m, 1H), 1.74 (m, 2H), 1.45 (m, 2H), 1.25 (dd, 2H), 1.09 (dd, 2H), 0.93 (t, 3H).

### Step B: Butyl 1-allylcyclopropane-1-sulfonate

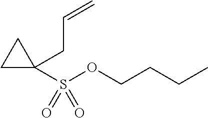

To a solution of 1-butyl cyclopropanesulfonate (4.8 g, 24.9 mmol) in THF at −78° C. was added simultaneously butyllithium solution (15.6 ml, 24.9 mmol, 1.6M, THF) and allyl iodide (24.9 mmol) under nitrogen atmosphere. The reaction mixture was stirred 2 hours at −78° C. and 3 hours at room temperature. The volatiles were evaporated under reduced pressure and the residue extracted with CH₂C2 (100 ml). The extract was washed with water, dried (MgSO₄) and evaporated. The residue was purified over silica gel chromatography (eluants: hexane/CH₂Cl₂) to obtain the titled product (3.75 g, 69.0%) as a colorless oil. ¹H NMR (300 MHz, CDCl₃): δ 5.6 (m, 1H), 5.13-5.08 (t, 2H), 4.21 (t, 2H), 2.65 (d, 2H), 1.7 (m, 2H), 1.4 (m, 4H), 0.93 (m, 5H).

### Step C: Potassium 1-allylcyclopropane-1-sulfonate

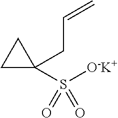

A mixture of 1-butyl 1-methyl-cyclopropanesulfonate (3.75 g, 17.2 mmol) and potassium thiocyanate (1.7 g, 17.2 mmol) in DME (20 ml) and water (20 ml) was refluxed for 16 h. The volatiles were evaporated to obtain the crude sulfonate (3.44 g, quantitative) which was dried under vacuum at 50° C. for 16 h. The crude product was used in the next reaction without further purification. ¹H NMR (CDCl₃): δ 5.6 (m, 1H), 4.91-4.85 (dd, 2H), 2.471-2.397 (d, 2H), 0.756 (m, 2H), 0.322 (m, 2H).

### Step D: L-allylcyclopropane-1-sulfonyl chloride

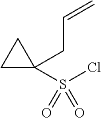

A solution of potassium l-allylcyclopropane-1-sulfonate (3.44 g, 17.2 mmol), thionyl chloride (10 ml) and DMF (5 drops) was refluxed at 60° C. for 16 h. The volatiles evaporated under reduced pressure and the residue extracted with CH₂Cl₂ (50 ml). The extract was washed with water, dried (MgSO₄) and evaporated to obtain the crude product as yellow gummy oil which was washed with hexane and used in the next reaction without further purification (2.7 g, 15 mmol, 87%). ¹HNMR (300 MHz, CDCl₃): δ 5.728 (m, 1H), 5.191 (t, 2H), 2.9 (d, 2H), 0.756 (m, 2H), 0.322 (m, 2H).

### Step E: 1-allyl-N-(3-difluoro-2-(2-fluoro-4-iodophenylamino)phenyl)cyclopropane-1-sulfonamide

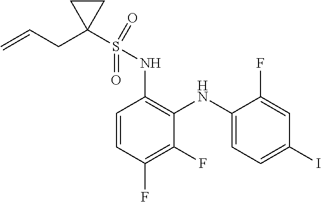 [SMILES: C=CCC1(S(=O)(=O)Nc2ccc(F)c(F)c2Nc2ccc(I)cc2F)CC1]

According to the general procedure B, 5,6-difluoro-N1-(2-fluoro-4-iodophenyl)benzene-1,2-diamine was reacted with 1-allylcyclopropane-1-sulfonyl chloride to obtain the desired product. m/z 507 [M-1]⁻.

### Step F: N-(3,4-difluoro-2-(2-fluoro-4-iodophenylamino)phenyl)-1-2,3-dihydroxypropyl)cyclopropane-1-sulfonamide

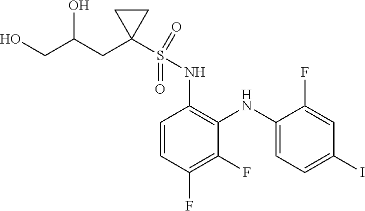 [SMILES: O=S(=O)(Nc1ccc(F)c(F)c1Nc1ccc(I)cc1F)C1(CC(O)CO)CC1]

1-Allyl-N-(3,4-difluoro-2-(2-fluoro-4-iodophenylamino)phenyl)cyclopropane-1-sulfonamide (0.77 g, 1.52 mmol) and 4-methylmorpholine N-oxide (0.18 g, 1.52 mmol) were dissolved in THF (50 mL). Osmium tetroxide was added at room temperature (0.152 mmol, 0.965 mL, 40% in H₂O) and the reaction mixture was stirred at room temperature for 16 hours. EtOAc was added, the organic phase was washed with water, dried (MgSO₄) and concentrated under reduced pressure. The residue was purified over silica gel chromatography (eluants: EtOAc/MeOH) to obtain the titled product (0.65 g, 79%). ¹H NMR (300 MHz, CDCl₃+D₂O): δ 7.38 (dd, J=1.8 & 10.5 Hz, 1H), 7.36 (ddd, J=2.4, 5.1 & 9.3 Hz, 1H), 7.25 (d, J=8.7 Hz, 1H), 7.02 (dd, J=9.0 & 17.7 Hz, 1H), 6.27 (dt, J=3.0, 8.7 & 17.4 Hz, 1H), 3.92 (m, 1H), 3.54 (dd, J=3.9 & 11.1 Hz, 1H), 3.39 (dd, J=6.6 & 11.1 Hz, 1H), 2.16 (dd, J=9.6 & 15.9 Hz, 1H), 1.59 (d, J=14.1 Hz, 1H), 1.41 (m, 1H), 1.26 (m, 1H), 0.83 (m, 2H); m/z 542 [M-1]⁻.

### Example 1A

### (S)—N-(3,4-difluoro-2-(2-fluoro-4-iodophenylamino)phenyl)-1-(2,3-dihydroxypropyl)cyclopropane-1-sulfonamide

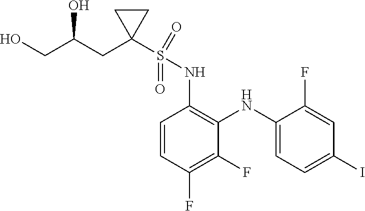 [SMILES: O=S(=O)(Nc1ccc(F)c(F)c1Nc1ccc(I)cc1F)C1(C[C@H](O)CO)CC1]

The pure S isomer was obtained by chiral HPLC separation of the racemic mixture (example 13). ¹H NMR (300 MHz, CDCl₃+D₂O): δ 7.38 (dd, J=1.8 & 10.5 Hz, 1H), 7.36 (ddd, J=2.4, 5.1 & 9.3 Hz, 1H), 7.25 (d, J=8.7 Hz, 1H), 7.02 (dd, J=9.0 & 17.7 Hz, 1H), 6.27 (dt, J=3.0, 8.7 & 17.4 Hz, 1H), 3.92 (m, 1H), 3.54 (dd, J=3.9 & 11.1 Hz, 1H), 3.39 (dd, J=6.6 & 11.1 Hz, 1H), 2.16 (dd, J=9.6 & 15.9 Hz, 1H), 1.59 (d, J=14.1 Hz, 1H), 1.41 (m, 1H), 1.26 (m, 1H), 0.83 (m, 2H); m/z=542 [M-1]⁻.

### Example 1B

### Example 1A

### (R)—N-(3,4-difluoro-2-(2-fluoro-4-iodophenylamino)phenyl)-1-(2,3-dihydroxypropyl)cyclopropane-1-sulfonamide

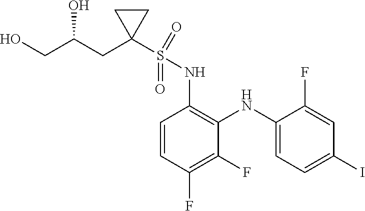 [SMILES: O=S(=O)(Nc1ccc(F)c(F)c1Nc1ccc(I)cc1F)C1(C[C@@H](O)CO)CC1]

The pure R isomer was obtained by chiral HPLC separation of the racemic mixture (example 13). ¹H NMR (300 MHz, CDCl₃+D₂O): δ 7.38 (dd, J=1.8 & 10.5 Hz, 1H), 7.36 (ddd, J=2.4, 5.1 & 9.3 Hz, 1H), 7.25 (d, J=8.7 Hz, 1H), 7.02 (dd, J=9.0 & 17.7 Hz, 1H), 6.27 (dt, J=3.0, 8.7 & 17.4 Hz, 1H), 3.92 (m, 1H), 3.54 (dd, J=3.9 & 11.1 Hz, 1H), 3.39 (dd, J=6.6 & 11.1 Hz, 1H), 2.16 (dd, J=9.6 & 15.9 Hz, 1H), 1.59 (d, J=14.1 Hz, 1H), 1.41 (m, 1H), 1.26 (m, 1H), 0.83 (m, 2H); m/z=542 [M-1]⁻.

### Example 2

### 1-(2,3-Dihydroxy-propyl)-cyclopropanesulfonic acid [3,4,6-trifluoro-2-(4-fluoro-2-iodo-phenylamino)-phenyl]-amide

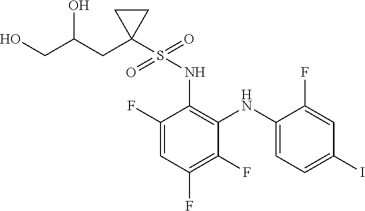 [SMILES: O=S(=O)(Nc1c(F)cc(F)c(F)c1Nc1ccc(I)cc1F)C1(CC(O)CO)CC1]

### Step A: 1-Allyl-cyclopropanesulfonic acid [3,4.6-trifluoro-2-(2-fluoro-4-iodo-phenylamino)phenyl]-amide

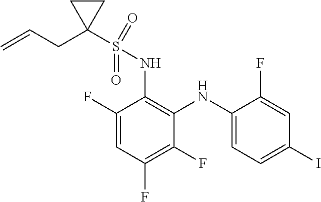 [SMILES: C=CCC1(S(=O)(=O)Nc2c(F)cc(F)c(F)c2Nc2ccc(I)cc2F)CC1]

To a stirred solution of the amine, i.e., 3,5,6-trifluoro-N¹-(2-fluoro-4-iodophenyl)benzene-1,2-diamine, (1 eq) in anhydrous pyridine (5 ml/mmole) was added the sulfonyl chloride, i.e., 1-allyl-cyclopropanesulfonyl chloride, (1-5 eq). The reaction mixture was stirred at 40° C. for 48 hours. The reaction mixture was partitioned with water and EtOAc. The organic layer was washed with brine, dried (MGSO₄) and concentrated under reduced pressure. The residue was purified by flash column chromatography on silica. ¹H NMR (CDCl₃, 300 MHz): δ 7.41 (dd, 1H), 7.38 (dd, 1H), 7.09 (s, 1H), 6.78 (m, 1H), 6.49 (m, 1H), 5.96 (s, 1H), 5.86 (m, 1H), 5.18 (d, 2H), 2.76 (d, 2H), 1.23 (m, 2H), 0.872 (m, 2H).

### Step B: 1-(2,3-Dihydroxypropyl-N-(3,4,6-trifluoro-2-2-fluoro-4-iodophenylamino)phenyl)cyclopropane-1-sulfonamide

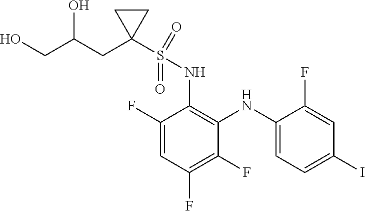 [SMILES: O=S(=O)(Nc1c(F)cc(F)c(F)c1Nc1ccc(I)cc1F)C1(CC(O)CO)CC1]

1-Allyl-cyclopropanesulfonic acid [3,4,6-trifluoro-2-(2-fluoro-4-iodo-phenyl amino)-phenyl]-amide (110 mg, 0.21 mmol) and 4-methylmorpholine N-oxide (24.6 mg, 0.21 mmol) was dissolved in THF (8 mL). Osmium tetroxide was added at room temperature (0.021 mmol, 0.153 mL, 4% in H₂O) and the reaction mixture was stirred at room temperature for 16 hours. EtOAc was added, the organic phase was washed with water, dried (MgSO₄) and concentrated under reduced pressure. The residue was purified over silica gel chromatography (eluants: EtOAc/MeOH) to obtain the titled product (0.89 g, 75%). ¹H NMR (CDCl₃, 300 MHz): δ 7.39 (dd, J=1.5 & 10.6 Hz, 1H), 7.29 (d, J=8.8 Hz, 1H), 7.28 (s, 1H), 6.97 (s, 1H), 6.76 (m, 1H), 6.49 (m, 1H), 4.13 (m, 1H), 3.66 (dd, J=3.7 & 11.4 Hz, 1H), 3.53 (dd. J=6.7 & 11.2 Hz, 1H), 2.50 (dd, J=10.0 & 16.1 Hz, 1H), 1.6 (m, 1H), 1.46 (m, 1H), 1.28 (m, 1H), 1.20 (m, 2H), 0.92 (m, 21H); m/z 559 [M-1]⁻.

### Example 2A

### (S)-1-(2,3-dihydroxypropyl)-N-(3,4,6-trifluoro-2-(2-fluoro-4-iodophenylamino)phenyl)cyclopropane-1-sulfonamide

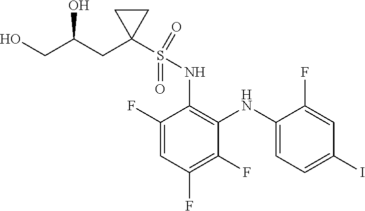 [SMILES: O=S(=O)(Nc1c(F)cc(F)c(F)c1Nc1ccc(I)cc1F)C1(C[C@H](O)CO)CC1]

The pure S isomer was obtained by chiral HPLC separation of the racemic mixture (example 52). ¹H NMR (CDCl₃, 300 MHz): δ 7.39 (dd, J=1.5 & 10.6 Hz₅ 1H), 7.29 (d, J=8.8 Hz, 1H), 7.28 (s, 1H), 6.97 (s, 1H), 6.76 (m, 1H), 6.49 (m, 1H), 4.13 (m, 1H), 3.66 (dd, J=3.7 & 11.4 Hz₅ 1H), 3.53 (dd, J=6.7 & 11.2 Hz, 1H), 2.50 (dd, J=10.0 & 16.1 Hz, 1H), 1.6 (m, 1H), 1.46 (m, 1H), 1.28 (m, 1H), 1.20 (m, 2H), 0.92 (m, 2H); m/z 559 [M-1]⁻.

### Example 2B

### (R)-1-(2,3-dihydroxypropyl)-N-(3,4,6-trifluoro-2-(2-fluoro-4-iodophenylamino)phenyl)cyclopropane-1-sulfonamide

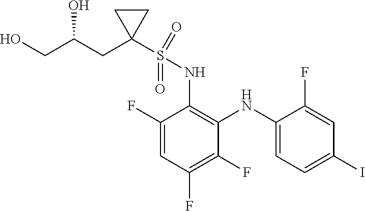 [SMILES: O=S(=O)(Nc1c(F)cc(F)c(F)c1Nc1ccc(I)cc1F)C1(C[C@@H](O)CO)CC1]

The pure R isomer was obtained by chiral HPLC separation of the racemic mixture (example 52). ¹H NMR (CDCl₃, 300 MHz): δ 7.39 (dd, J=1.5 & 10.6 Hz, 1H), 7.29 (d, J=8.8 Hz, 1H), 7.28 (s, 1H), 6.97 (s, 1H), 6.76 (m, 1H), 6.49 (m, 1H), 4.13 (m, 1H), 3.66 (dd, J=3.7 & 11.4 Hz, 1H), 3.53 (dd, J=6.7 & 11.2 Hz, 1H), 2.50 (dd, J=10.0 & 16.1 Hz, 1H), 1.6 (m, 1H), 1.46 (m, 1H), 1.28 (m, 1H), 1.20 (m, 2H), 0.92 (m, 2H); m/z=559 [M-1]⁻.

### Example 3

### Synthesis of N-(4-(2-fluoro-4-iodophenylamino)-1,5-dimethyl-6-oxo-1,6-dihydropyridin-3-yl)cyclopropanesulfonamide

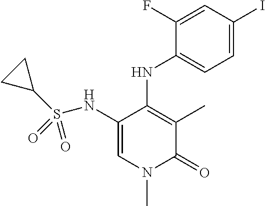 [SMILES: Cc1c(Nc2ccc(I)cc2F)c(NS(=O)(=O)C2CC2)cn(C)c1=O]

### Step a: Diethyl 2-methyl-3-oxopentanedioate

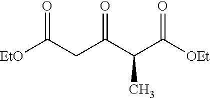

This compound was synthesized according to U.S. Pat. No. 6,833,471. To 20 mL of dry THF that had been purged with Ar (gas) was added diethyl 3-oxopentanedioate (5 mL, 27.54 mmol) and the solution was cooled to −15° C. prior to the dropwise addition of LDA (2M) (15 mL, 30 mmol). The reaction was maintained under Ar (gas) at −15° C., and MeI (3 mL, 48.2 mmol) was added slowly. The reaction was allowed to reach room temperature gradually over 3 hours, and the stirring was continued overnight. After 18 hours, the reaction mixture was poured into 140 mL of a 1:1 mixture of 0.5 N HCl (aq) and Et₂O. The organic layer was separated, and the aqueous layer was extracted twice with Et₂O (15 mL×2). The organic layers were combined, washed with brine, dried (MgSO₄) and concentrated to give an yellow oil, which was flash chromatography purified (SiO₂, Hexane:EtOAc=8:2 (v:v)) to afford a colorless/light yellow oil as the title compound. (1.37 g, 23% yield). MW m/z: 215.3 (MW−1, low intensity). ¹H NMR (CDCl₃, 300 Hz) δ ppm 4.20 (q, 4H), 3.68 (q, 1H), 3.60 (dd, 2H), 1.37 (d, 3H), 1.26 (t, 6H).

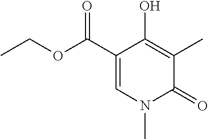

### Step b: Ethyl 4-hydroxy-1,5-dimethyl-6-oxo-1,6-dihydropyridine-3-carboxylate

Triethyl orthoformate (1.25 mL, 7.51 mmol) and Ac₂O (2 mL) were added to diethyl 2-methyl-3-oxopentanedioate (1.37 g, 6.34 mmol) and heated to 135° C. After 1.5 hours, the reaction mixture was cooled to room temperature and concentrated under the reduced pressure. The resulting residue was cooled to 0° C. under an ice-water bath, and MeNH₂ (40% in water) (3 mL) was added. The resulting mixture was stirred at room temperature for 16 hours. Aqueous HCL (1N) was added until pH˜7. The solution was extracted with EtOAc (30 mL×2). The combined organic layers were washed with brine, dried (MgSO₄) and concentrated to give a solid, which was purified by flash chromatograph (SiO₂, EtOAc:DCM=1:1 (v:v), Rf˜0.4) to afford an off-white solid as the title compound. (314 mg, 23% yield). MW m/z: 212.2 (MW+1), 234.2 (MW+Na); 210.2 (MW−1). ¹H NMR (DMSO-d6, 300 Hz): δ ppm 10.71 (s, br, 1H), 8.46 (s, 1H), 4.32 (q, J=7.2 Hz, 2H), 3.45 (s, 3H), 1.83 (s, 3H), 1.30 (t, J=7.2 Hz 3H).

### Step c: 4-Chloro-1,5-dimethyl-6-oxo-1,6-dihydropyridine-3-carboxylate

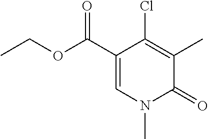

To the mixture of ethyl 4-hydroxy-1,5-dimethyl-6-oxo-1,6-dihydropyridine-3-carboxylate (310 mg, 1.47 mmol) dissolved in dry toluene (13 mL) was added POCl₃ (600 uL, 6.44 mmol). The resulted mixture was heated to 110° C. for 3 hours. After cooled to room temperature, the mixture was poured into ice-cold saturated aqueous NaHCO₃ (50 mL) to make it basic. The mixture was extracted with EtOAc (50 mL×2). The organic layers were combined, washed with brine, dried (MgSO₄) and concentrated to give a brown solid, which was purified by TLC (SiO₂, EtOAc:DCM=6:4 v:v; Rf˜0.6) to afford an off-white solid as the title compound. (178 mg, 53% yield). MW m/z: 231.3 (MW−1); 227.8 (MW−1). ¹H NMR (DMSO-d6, 300 Hz): δ ppm 8.04 (s, 1H), 4.33 (q, J=7.2 Hz, 2H), 3.59 (s, 3H), 2.27 (s, 3H), 1.37 (t, J=7.2 Hz, 3H).

### Step d: 4-Chloro-1,5-dimethyl-6-oxo-1,6-dihydropyridine-3-carboxylic acid

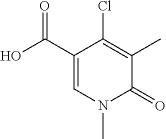

To a solution of ethyl 4-chloro-1,5-dimethyl-6-oxo-1,6-dihydropyridine-3-carboxylate (172 mg, 0.75 mmol) dissolved in a 4:1 mixture of THF:MeOH (5 mL) (v:v), was added a aqueous solution of LiOH (1.52 mmol, 1M). After stirring for 40 min, the reaction mixture was acidified to pH˜1 with HCl (1N, aq) and extracted with EtOAc (30 mL x 3). The combined organic layers were washed with brine (30 mL), dried (MgSO4), filtered and concentrated under the reduced pressure to give an off-white solid as the title compound. (163 mg, 100% yield).

MW m/z: 202.3 (MW+1), 204.2 (MW−1+C1 pattern); 200.4 (MW−1), 202.4 (MW−1+C1 pattern).

¹H NMR (DMSO-d6, 300 Hz): δ ppm 12.97 (s, 1H), 8.42 (s, 1H), 3.48 (s, 3H), 2.10 (s, 3H).

### Step e: 4-(2-Fluoro-4-iodophenylamino)-1,5-dimethyl-6-oxo-1,6-dihydropyridine-3-carboxylic acid

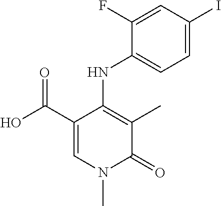 [SMILES: Cc1c(Nc2ccc(I)cc2F)c(C(=O)O)cn(C)c1=O]

To the stirred solution of 2-fluoro-4-iodoaniline (470 mg, 1.94 mmol) in dry THF (4 mL) cooled to −78° C., was added LDA (2M in THF) (1.35 mL, 2.70 mmol). After vigorous stirring for 10 minutes at this temperature, a solution of 4-chloro-1,5-dimethyl-6-oxo-1,6-dihydropyridine-3-carboxylic acid (160 mg, 0.792 mmol) dissolved in dry THF (8 mL) was added dropwise through a syringe. The dry-ice bath was removed after 1 hour, and the reaction was stirred for 16 hours at room temperature. At this time, LC/MS indicated 23% of the title product and 33% of unreacted chloride in the reaction mixture. The same reaction mixture was continued to stir at room temperature for additional 24 hours. The mixture was then re-cooled to −78° C. under a dry-ice/acetone bath. Additional LDA (1.35 mL, 2.70 mmol) (2M in THF) was added to the reaction mixture and slowly warmed to room temperature in 16 hours until LC/MS showed the consumption of chloride material. The mixture was cooled to −5° C., and aqueous HCl (1N) (15 mL) was added. The solution was extracted with EtOAc (15 mL×3). The combined organic layers was dried (MgSO₄) and concentrated to give a residue which was triturated with DCM to give a solid. The title compound was used for the next reaction without further purification. (165 mg, 52% yield). MW m/z: 403.13 (MW+1), 401.18 (MW−1). ¹H NMR (DMSO-d6, 300 Hz): δ ppm 13.26 (s, br, 1H), 9.08 (s, 1H), 8.48 (s, 1H), 7.62 (d, J=10.8 Hz, 1H), 7.39 (d, J=8.1 Hz, 1H), 6.49 (t. J=8.7 Hz, 1H), 3.48 (s, 3H), 1.58 (s, 3H)

### Step f: 1-(2-Fluoro-4-iodophenyl)-5,7-dimethyl-1H-imidazo[4,5-c]pyridine-2,6 (3H,5H)-dione

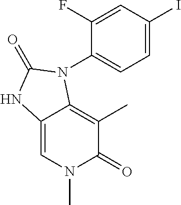 [SMILES: Cc1c(=O)n(C)cc2[nH]c(=O)n(-c3ccc(I)cc3F)c12]

To the suspension of 4-(2-fluoro-4-iodophenylamino)-1,5-dimethyl-6-oxo-1,6-dihydropyridine-3-carboxylic acid (148 mg, 0.368 mmol) in dry toluene (15 mL), was added DPPA (95 uL, 0.439 mmol) and followed by TEA (56 uL, 0.40 mmol). The solution became clear pink and was heated to 100° C. under Argon for 4 hours, at which time LC/MS indicated the complete disappearance of starting material. Aqueous HCl (1N) (25 mL) was added, and the solution was extracted with EtOAc (15 mL×3). The combined organic layers was washed with brine, dried (MgSO₄), and concentrated to give an oil residue, which was purified via flash chromatography (SiO₂, EtOAc:MeOH 9:1, Rf˜0.25) to give an off-white solid as the title compound. (139 mg, 95% yield). MW m/z: 400.1 (MW+1), 398.2 (MW−1). ¹H NMR (DMSO-d6, 300 Hz): δ ppm 10.95 (s, 1H), 7.90 (dd, J=9.6 Hz, 1H), 7.73 (d, J=8.4 Hz, 1H), 7.36 (t, J=8.4 Hz, 1H), 7.35 (s, 1H), 3.40 (s, 3H), 1.47 (s, 3H)

### Step g: N-(4-(2-fluoro-4-iodophenylamino)-1,5-dimethyl-6-oxo-1,6-dihydropyridin-3-yl)cyclopropanesulfonamide

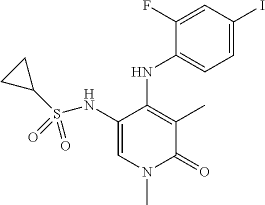 [SMILES: Cc1c(Nc2ccc(I)cc2F)c(NS(=O)(=O)C2CC2)cn(C)c1=O]

To the solution of 1-(2-fluoro-4-iodophenyl)-5,7-dimethyl-1H-imidazo[4,5-c]pyridine-2,6(3H,5H)-dione (23 mg, 0.0576) dissolved in dry DMF (2 mL) cooled to below 0° C. under an ice-bath, was added NaH (60% in mineral oil) (5.0 mg, 0.125 mmol). The cooling bath was removed after addition and the solution was allowed to stir at room temperature for 1 hour. The same solution was re-cooled to −5° C. in a dry-ice/acetone bath, and added cyclopropanesulfonyl chloride (28 mg, 0.20 mmol) dissolved in dry THF (0.5 mL) slowly. The mixture was allowed to warm to room temperature and stirred was and additional 16 hours. The reaction mixture was cooled to 0° C., additional NaH (60% in oil) (5.0 mg, 0.125 mmol), followed by cyclopropanesulfonyl chloride (15 mg, 0.11 mmol) were added. The solution was stirred at room temperature for additional 5 hours. To the same reaction mixture was added aqueous NaOH (1N) (5 mL). The mixture was heated to 65° C. for 40 minutes. After cooled to room temperature, aqueous HCl (1N) (25 mL) was added to acidify the solution, which was extracted with EtOAc (15 mL×3). The combined organic layers was washed with brine, dried (MgSO₄), and concentrated under the reduced pressure to give a residue, which was HPLC purified. (9.6 mg, 35% yield). MW m/z: 478.08 (MW+1), 476.10 (MW−1). ¹H NMR (DMSO-d6, 300 Hz): δ ppm 8.89 (s, 1H), 7.65 (s, 1H), 7.56 (dd, J=10.8, 1.5 Hz, 1H), 7.42 (s, 1H), 7.0 (d, J=8.7 Hz, 1H), 6.34 (t, J=8.7 Hz, 1H), 3.43 (s, 3H), 2.43 (m, 2H), 1.65 (s, 3H), 0.69-0.79 (m, 4H)

### Example 4

### Synthesis of N-(3,4-difluoro-2-(2-fluoro-4-iodophenylamino)phenyl)-1-(2,3-dihydroxypropyl)cyclopropane-1-sulfonamide

### Step A: 1-Allyl-N-(3,4-difluoro-2-(2-fluoro-4-iodophenylamino)-6-methoxyphenyl)cyclopropane-1-sulfonamide

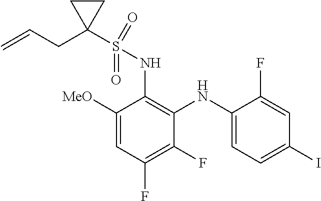 [SMILES: C=CCC1(S(=O)(=O)Nc2c(OC)cc(F)c(F)c2Nc2ccc(I)cc2F)CC1]

According to the general procedure B, 1-allyl-cyclopropanesulfonyl chloride was reacted with 5,6-difluoro-N1-(2-fluoro-4-iodophenyl)-3-methoxybenzene-1,2-diamine to obtain the title product. 1H NMR (CDCl₃, 300 MHz): δ 7.417 (dd, 1H), 7.309 (s, 1H), 7.25 (m, 1H), 6.89 (m, 1H), 6.52 (m, 1H), 6.427 (m, 1H), 6.03 (s, 1H), 5.668 (m, 1H), 5.11 (t, 1H), 3.9 (s, 3H), 2.75 (d, 2H), 1.21 (m, 2H), 0.767 (m, 2H).

### Step B: N-(3,4-difluoro-2-(2-fluoro-4-iodophenylamino)phenyl)-1-(2,3-dihydroxypropyl)cyclopropane-1-sulfonamide

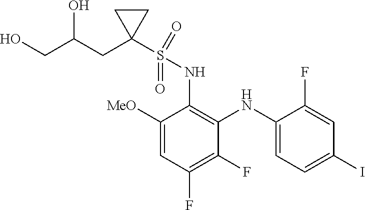 [SMILES: COc1cc(F)c(F)c(Nc2ccc(I)cc2F)c1NS(=O)(=O)C1(CC(O)CO)CC1]

1-Allyl-N-(3,4-difluoro-2-(2-fluoro-4-iodophenylamino)-6-methoxyphenyl)cyclopropane-1-sulfonamide (97 mg, 0.18 mmole) and 4-methylmorpholine N-oxide (21 mg, 0.18 mmole) were dissolved in THF (8 mL). Osmium tetroxide was added at room temperature (0.018 mmole, 0.13 mL, 4% in H₂O) and the reaction mixture was stirred at room temperature for 16 hours. EtOAc was added, the organic phase was washed with water, dried (MgSO₄) and concentrated under reduced pressure. The residue was purified over silica gel chromatography (eluants: EtOAc/MeOH) to obtain the titled product (0.80 g, 78%). ¹H NMR (CDCl₃, 300 MHz): δ 7.38 (dd, J=1.7 & 10.3 Hz, 1H), 7.26 (m, 1H), 7.14 (s, 1H), 6.87 (s, 1H), 6.53 (dd, J=6.8 & 11.4 Hz, 1H), 6.43 (m, 1H), 4.06 (m, 1H), 3.89 (s, 3H), 3.63 (dd, J 3.7 & 11.1 Hz, 1H), 3.49 (dd, J=6.4 & 11.1 Hz, 1H), 2.3 (dd, J=9.7 & 16.1 Hz, 1H), 1.77 (dd, J=1.9 & 16.0 Hz, 1H), 1.37 (m, 1H), 1.25 (m, 1H), 1.21 (m, 2H), 0.86 (m, 2H); m/z=571 [M-1]⁻.

### Example 5

### Synthesis of N-(3,4-difluoro-2-(2-fluoro-4-iodophenylamino)-6-methoxyphenyl)-1-(2-hydroxyethyl)cyclopropane-1-sulfonamide

### Step A: TBS-protected N-(3,4-difluoro-2-2-fluoro-4-iodophenylamino 6-methoxyphenyl)-1-(2-hydroxyethyl)cyclopropane-1-sulfonamide

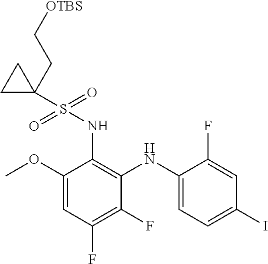 [SMILES: COc1cc(F)c(F)c(Nc2ccc(I)cc2F)c1NS(=O)(=O)C1(CCO[Si](C)(C)C(C)(C)C)CC1]

According to the general procedure B, the sulfonyl chloride was reacted with 5,6-difluoro-N1-(2-fluoro-4-iodophenyl)-3-methoxy-benzene-1,2-diamine to obtain the title product. Yield: 37%. ¹H-NMR (300 MHz, CDCl₃): δ 7.40-7.34 (dd, 1H), 7.23-7.21 (m, 1H), 6.61 (s, 1H, br), 6.57-6.49 (dd, 1H), 6.48-6.39 (m, 1H), 3.9-3.7 (m, 5H), 2.15-2.05 (t, 2H), 1.30-1.20 (m, 2H), 0.95-0.80 (m, 11H), 0.05 (s, 6H): m/z=655 [M-1]⁻.

### Step B: N-(3,4-difluoro-2-(2-fluoro-4-iodophenylamino)-6-methoxyphenyl)-1-(2-hydroxyethyl)cyclopropane-1-sulfonamide

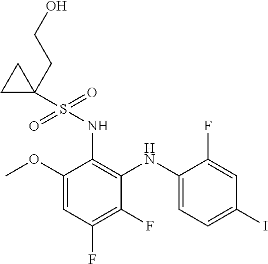 [SMILES: COc1cc(F)c(F)c(Nc2ccc(I)cc2F)c1NS(=O)(=O)C1(CCO)CC1]

Yield: 100%. ¹H-NMR (300 MHz, CDCl₃): δ=7.40-7.34 (dd, 1H), 7.23-7.21 (m, 1H), 6.61 (s, 1H, br), 6.57-6.49 (dd, 1H), 6.48-6.39 (m, 1H), 3.9-3.7 (m, 5H), 2.15-2.05 (t, 2H), 1.30-1.20 (m, 2H), 0.95-0.80 (m, 2H); m/z 541 [M-1]⁻.

### Example 6

### Synthesis of (S)—N-(3,4-difluoro-2-(2-fluoro-4-iodophenylamino)-6-methoxyphenyl)-1-(2,3-dihydroxypropyl)cyclopropane-1-sulfonamide

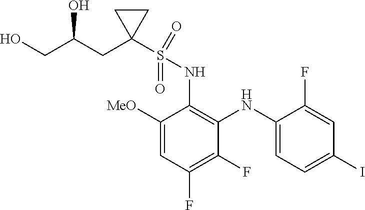 [SMILES: COc1cc(F)c(F)c(Nc2ccc(I)cc2F)c1NS(=O)(=O)C1(C[C@H](O)CO)CC1]

The pure S isomer was obtained by chiral HPLC separation of the racemic mixture (example 5) (¹H NMR (CDCl₃, 300 MHz): δ 7.38 (dd, J=1.7 & 10.3 Hz, 1H), 7.26 (m, 1H), 7.14 (s, 1H), 6.87 (s, 1H), 6.53 (dd, J=6.8 & 11.4 Hz, 1H), 6.43 (m, 1H), 4.06 (m, 1H), 3.89 (s, 3H), 3.63 (dd, J=3.7 & 11.1 Hz, 1H), 3.49 (dd, J=6.4 & 11.1 Hz, 1H), 2.3 (dd, J=9.7 & 16.1 Hz, 1H), 1.77 (dd, J=1.9 & 16.0 Hz, 1H), 1.37 (m, 1H), 1.25 (m, 1H), 1.21 (m, 2H), 0.86 (m, 2H); m/z=571 [M-1]⁻.

### Example 7

### Synthesis of (R)—N-(3,4-difluoro-2-(2-fluoro-4-iodophenylamino)-6-methoxyphenyl)-1-(2,3-dihydroxypropyl)cyclopropane-1-sulfonamide

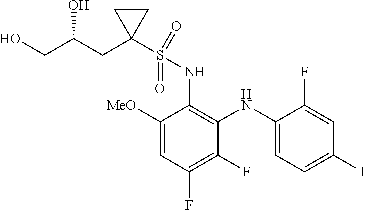 [SMILES: COc1cc(F)c(F)c(Nc2ccc(I)cc2F)c1NS(=O)(=O)C1(C[C@@H](O)CO)CC1]

The pure R isomer was obtained by chiral HPLC separation of the racemic mixture (example 5). 1H NMR (CDCl₃, 300 MHz): δ 7.38 (dd, J=1.7 & 10.3 Hz, 1H), 7.26 (m, 1H), 7.14 (s, 1H), 6.87 (s, 1H), 6.53 (dd, J=6.8 & 11.4 Hz, 1H), 6.43 (m, 1H), 4.06 (m, 1H), 3.89 (s, 3H), 3.63 (dd, J=3.7 & 11.1 Hz, 1H), 3.49 (dd, J=6.4 & 11.1 Hz, 1H), 2.3 (dd, J=9.7 & 16.1 Hz, 1H), 1.77 (dd, J=1.9 & 16.0 Hz, 1H), 1.37 (m, 1H), 1.25 (m, 1H), 1.21 (m, 2H), 0.86 (m, 2H); m/z=571 [M-1]⁻.

### Biology

### Study Design

The study was a single-arm, open-label, multicenter Phase II study. Patients were enrolled from 14 centers in South Korea, Taiwan, Hong-Kong, and Singapore.

The patient inclusion criteria included:
diagnosis of unresectable advanced or metastatic HCC; Child-Pugh A status; Eastern Cooperative Oncology Group performance status (ECOG PS) 0 or 1; ≧18 years of age; ≧1 untreated, unidimensional measurable lesion.

The patient exclusion criteria included:
previous treatment with either BAY 86-9766 or Sorafenib; prior systemic anticancer therapy for HCC; any previous or concurrent cancer ≦3 years prior to study entry.

### Dosage and Administration

The eligible patients received (S)—N-(3,4-difluoro-2-(2-fluoro-4-iodophenylamino)-6-methoxyphenyl)-1-(2,3-dihydroxypropyl)cyclopropane-1-sulfonamide [Compound 1](50 mg orally) twice daily in combination with oral Sorafenib (600 mg daily in cycle 1: 200 mg in the morning and 400 mg in the evening)
In cycle 2, if no hand-foot skin reaction, fatigue, or gastrointestinal toxicities of grade ≧2 occurred, Sorafenib dosing was escalated to 400 mg twice daily

The treatment was administered on a continuous basis until disease progression (PD), clinical progression, or other criterion for discontinuation of treatment was reached. Dose modifications were performed if clinically significant hematologic or other drug-related toxicities were reported.

### Efficacy Assessments

The primary efficacy variable was the disease control rate (DCR), defined as the proportion of patients who have a best response rating over the duration of the study of complete response, partial response, or stable disease, according to Response Evaluation Criteria in Solid Tumors (RECIST) version 1.1. Secondary efficacy variables included time to PD and OS. Tumor evaluation was performed at screening and every 6 weeks during treatment (beginning within the last 10 days of cycle 2) until PD or end of study treatment.

### KRAS and NRAS Mutation Detection

5 different genes were analyzed: BRAF, CSF-1R, KRAS, NRAS and PIK3CA. Mutations were evaluated in plasma patient s collected from 18 patients of the 26 (23+3) see table 3.

Several methods for detecting mutated-type RAS gene or protein are known and available on the market e.g. Cobas® KRAS Mutation Test marketed by Roche. Other methods are discussed in following publications:
Diehl F, Li M, He Y, Kinzler K W, Vogelstein B, Dressman D. (2006) BEAMing: single-molecule PCR on micoparticles in water-in-oil Emulsions. Nat Methods. 2006 July; 3(7):551-9 and
Diehl F., Schmidt K., Choti M. A., Romans K., Goodman S., Li M., Thornton K., Agrawal N., Sokoll L., Szabo S. A., Kinzler K. W., Vogelstein B., Diaz L. A. Jr. (2008) Circulating mutant DNA to assess tumor dynamics. Nature Medicine 14, 985-90.

### Results

### Patient Demographic and Disease Characteristics at Baseline

Of 95 patients enrolled in the study, 70 were assigned to study treatment,
All patients were of Asian race and the majority were male (86%) (Table 2),
Mean age at enrollment was 55 years. Almost 75% of patients were aged ≦65 years.

TABLE 2
Patient demographic and baseline characteristics

| | Total (N = 70) | |
|---|---|---|
| Gender, n (%) | | |
| Female | 10 | (14) |
| Male | 60 | (86) |
| Mean age at enrollment, years | 55 ± 12 | (28-78) |
| Age group, n (%) | | |
| ≦65 years | 52 | (74) |
| >65 years | 18 | (26) |

SD, standard deviation

### Efficacy of Compound 1 and Sorafenib Therapy

Of those patients, 23 (40%) had stable disease (≧10 weeks) and three (5%) had a confirmed partial response, resulting in an overall DCR of 45% (Table 3).

TABLE 3
Best overall response according to RECIST criteria
(primary efficacy analysis population)

| n (%) | Total (N = 58) | |
|---|---|---|
| Partial response | 3 | (5) |
| Stable disease | 23 | (40) |
| Unconfirmed partial response | 1 | (2) |
| Unconfirmed stable disease | 12 | (21) |
| Progressive disease | 14 | (24) |
| Not applicable | 1 | (2) |
| Missing | 4 | (7) |
| Primary end point: DCR | 26 | (45) |

18 patients of the 26 (23+3) were randomly tested. RAs mutations were detected in 3 of 18 plasma samples: KRAS G12A, KRAS G12R and NRAS Q61K. All 3 patients having received the combination comprising Compound 1 and Sorafenib show a long and durable partial response.

No mutation was identified for BRAF, PIK3CA or CSF-1R genes.

What is claimed is:

1. Use of one or more biomarkers defined as mutated RAS for predicting the pharmaceutical efficacy or clinical response of a combination comprising a MEK protein kinase inhibitor and Sorafenib or Regorafenib to be administered to a HCC patient.

2. The use of one biomarker defined as RAS gene or RAS protein according to claim 1.

3. The use according to claim 1 or 2 wherein RAS is KRAS, NRAS or HRAS.

4. The use according to claim 3 wherein RAS is KRAS or NRAS.

5. The use according to claims 1 to 4 wherein the MEK protein kinase inhibitor is a compound of formula A, or a pharmaceutically acceptable salt, solvate, polymorph, ester, amide, tautomer or prodrug thereof: 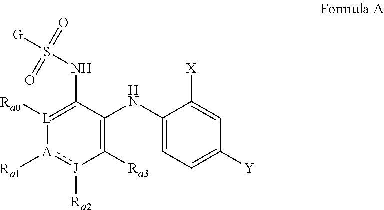
wherein
G is G₁, G₂, R₁ₐ, R_{1b}, R_{1c}, R_{1d}, R₁ₑ, Ar₁, Ar₂ or Ar₃;
Rₐ₀, R₁ and R₂ are independently selected from H, halogen, cyano, cyanomethyl, nitro, difluoromethoxy, difluoromethoxy, trifluoromethyl, azido, amino, alkylamino, dialkylamino, CO₂R₅, OR₅, —O—(CO)—R₅, —O—C(O)—N(R₅)₂, —NR₅C(O)NR₆R₇, —SR₅, NHC(O)R₅, —NHSO₂R₅, SO₂N(R₅)₂, C1-C6 alkyl, C1-C4 alkoxy, C3-C6 cycloalkyl, C2-C6 alkenyl, C2-C6 alkynyl, aryl, alkylaryl, arylalkyl, and heterocyclic;
each R₅ is selected from H, lower alkyl, substituted lower alkyl, aryl, or substituted aryl, and NR₇R₆;
wherein each R₆ and R₇ is independently selected from hydrogen or lower alkyl; wherein
said alkyl, cycloalkyl, alkenyl, aryl, alkylaryl, arylalkyl, heterocyclic and alkynyl groups are optionally substituted with 1-3 substituents selected independently from halogen, OH, CN, cyanomethyl, nitro, phenyl, difluoromethoxy, difluoromethoxy, and trifluoromethyl;
said C1-C6 alkyl and C1-C4 alkoxy groups are optionally substituted with OCH₃ or OCH₂CH₃;
Rₐ₁ is H, C₁-C₅ alkyl, C₃-C₆ cycloalkyl, C₂-C₆ alkenyl, C₅-C₆ cycloalkenyl or C₂-C₆ alkynyl;
wherein each alkyl, cycloalkyl, alkenyl, cycloalkenyl or alkynyl group is optionally substituted with 1-3 substituents selected independently from halogen, hydroxy, C₁-C₄ alky, C₁-C₄ alkoxy, cyano, cyanomethyl, nitro, azido, trifluoromethyl difluoromethoxy and phenyl, and
one or two ring carbon atoms of said C₃-C₆ cycloalkyl groups are optionally replaced with, independently, O, N, or S; or
Rₐ₁ is a 5 or 6-atom heterocyclic group, which group may be saturated, unsaturated, or aromatic, containing 1-5 heteroatoms selected independently from O, N, and S, which heterocyclic group is optionally substituted with 1-3 substituents selected independently from halogen, hydroxy, C₁-C₄ alky, C₁-C₄ alkoxy, cyano, cyanomethyl, nitro, azido, trifluoromethyl difluoromethoxy and phenyl;
Rₐ₂ is H, halogen, F, or oxo; or
Rₐ₁ and Rₐ₂, taken together, are -Q(R₂)—U(R₁)=D-
Rₐ₃ is H, halogen, hydroxy, azido, cyano, cyanomethy, C₁-C₆ alkyl, C₃-C₆ cycloalkyl, C₂-C₆ alkenyl, C₅-C₆ cycloalkenyl or C₂-C₆ alkynyl, wherein each alkyl, cycloalkyl, alkenyl cycloalkenyl or alkynyl group is optionally substituted with 1-3 substituents selected independently from halogen, hydroxy, C₁-C₄ alkoxy, cyano, cyanomethyl, nitro, azido, trifluoromethyl and phenyl; [symbol]
is a single or a double bond;
X and Y are independently selected from F, I, Br, Cl, CF₃, C1-C3 alkyl, C2-C3 alkenyl, C2-C3 alkynyl, cyclopropyl, phenyl, pyridyl, pyrazolyl, OMe, OEt, or SMe, or Het, where Het is a 5- to 10-membered mono- or bicyclic heterocyclic group, which group is saturated, olefinic, or aromatic, containing 1-5 ring heteroatoms selected independently from N, 0, and S; where
all said phenyl or Het groups are optionally substituted with F, Cl, Br, I, acetyl, methyl, CN, NO₂, CO₂H, C₁-C₃ alkyl, C₁-C₃ alkoxy, C₁-C₃ alkyl-C(═O)—, C₁-C₃ alkyl-C(═S)—, C₁-C₃ alkoxy-C(═S)—, C₁-C₃ alkyl-C(═O)O—, C₁-C₃ alkyl-O—(C═O)—, C₁-C₃ alkyl-C(═O)NH—, C₁-C₃ alkyl-C(═NH)NH—, C₁-C₃ alkyl-NH—(C═O)—, di-C₁-C₃ alkyl-N—(C═O)—, C₁-C₃ alkyl-C(═O)N(C₁-C₃ alkyl)-, C₁-C₃ alkyl-S(═O)₂NH— or trifluoromethyl;
all said methyl, ethyl, C1-C3 alkyl, and cyclopropyl groups of X and Y are optionally substituted with OH;
all said phenyl, pyridyl, pyrazolyl groups of Y are optionally substituted with halogen, acetyl, methyl, and trifluoromethyl; and
all said methyl groups of X and Y are optionally substituted with one, two, or three F atoms;
A, D, J, L, Q, U are independently selected from C, CH, —NH, N, O, and —N(CH₃)—;
G₁ is C₁-C₆ alkyl optionally substituted with one amino, C₁-C₃ alkylamino, or dialkylamino group, said dialkylamino group comprising two C₁-C₄ alkyl groups which may be identical or non-identical; or
G₁ is a C₃-C₅ diamino alkyl group;
G₂ is a 5- or 6-membered ring, which is saturated, unsaturated, or aromatic, containing 1-3 ring heteroatoms selected independently from N, O, and S, optionally substituted with 1-3 substituents selected independently from F, Cl, OH, O(C₁-C₃ alkyl), OCH₃, OCH₂CH₃, CH₃C(═O)NH, CH₃C(═O)O, CN, CF₃, and a 5-membered aromatic heterocyclic group containing 1-4 ring heteroatoms selected independently from N, O, and S;
R₁ₐ is methyl, cyclopropoxy or C1-C4 alkoxy; wherein
the methyl is optionally substituted with OH, 1-3 fluorine atoms or 1-3 chlorine atoms;
the C1-C4 alkyl moieties of said C1-C4 alkoxy are optionally substituted with one hydroxy or methoxy group; and
all C2-C4 alkyl groups within said C1-C4 alkoxy are optionally further substituted with a second OH group;
R_{1b} is CH(CH₃)—C1-3 alkyl or C3-C6 cycloalkyl, said CH₃, alkyl, and cycloalkyl groups optionally substituted with 1-3 substituents selected independently from F, Cl, Br, I, OH, C1-C4 alkoxy and CN;
R_{1c} is (CH₂)ₙOₘR′, where
m is 0 or 1;
n is 0, 1, 2, or 3;
R′ is C1-C6 alkyl, optionally substituted with 1-3 substituents selected independently from F, Cl, OH, OCH₃, OCH₂CH₃, and C3-C6 cycloalkyl;
R_{1d} is C(A′)(A″)(B)— wherein
B, A′, and A″ are, independently, H, substituted or unsubstituted C1-6 alkyl, substituted or unsubstituted C2-6 alkenyl, or
A′ and A″, together with the carbon atom to which they are attached, form a substituted or unsubstituted 3- to 6-member saturated ring;
R₁ₑ is benzyl or 2-phenyl ethyl, in which the phenyl group is optionally substituted 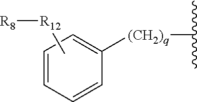
where
q is 1 or 2;
R₈ and R₉ are, independently, H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl;
R₁₀ is H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl, nitro, acetamido, amidinyl, cyano, carbamoyl, methylcarbamoyl, dimethylcarbamoyl, 1,3,4-oxadiazol-2-yl, 5-methyl-1,3,4-5 oxadiazolyl, 1,3,4-thiadiazolyl, 5-methyl-1,3,4-thiadiazol-1H-tetrazolyl, N-morpholinyl carbonylamino, N-morpholinylsulfonyl or N-pyrrolidinylcarbonylamino;
R₁₁ and R₁₂ are, independently, H, F, Cl, or methyl;
Ar₁ is 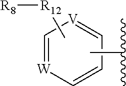
where
W and V are, independently, N, CR₈ or CR₉;
R₈, R₉ and R₁₀ are, independently, H, F, Cl, Br, CH₃, CH₂F, CHF₂, CF₃, OCH₃, OCH₂F, OCHF₂, OCF₃, ethyl, n-propyl, isopropyl, cyclopropyl, isobutyl, sec-butyl, tert-butyl, and methylsulfonyl, nitro, acetamido, amidinyl, cyano, carbamoyl, methylcarbamoyl, dimethylcarbamoyl, 1,3,4-oxadiazol-2-yl, 5-methyl-1,3,4-oxadiazol, 1,3,4-thiadiazol, 5-methyl-1,3,4-thiadiazol, 1H-tetrazolyl, N-morpholinylcarbonylamino, N-morpholinylsulfonyl and N-pyrrolidinylcarbonylamino;
R₁₁ and R₁₂ are, independently, H, F, Cl or methyl;
Ar₂ is 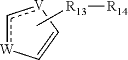
where
the dashed line represents a double bond which may be located formally either between V and the carbon between W and V, or between W and the carbon between W and V;
W is —S—, —O— or —N═, wherein
when W is —O— or —S—, V is —CH, —CCl═ or —N═; and
when W is —N═, V is CH, CCl, N or —NCH₃—;
R₁₃ and R₁₄ are, independently, H, methoxycarbonyl, methylcarbamoyl, acetamido, acetyl, methyl, ethyl, trifluoromethyl or halogen;
Ar₃ is 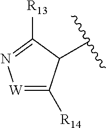
where
W is —NH—, —NCH₃— or —O—; and
R₁₃ and R₁₄ are, independently, H, F, Cl, or methyl.

6. The use according to claims 1 to 5 wherein the combination comprises the MEK protein kinase having the following structure: 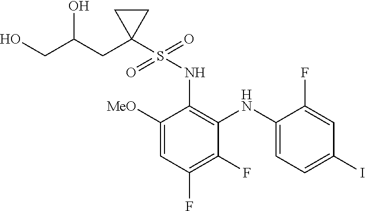 [SMILES: COc1cc(F)c(F)c(Nc2ccc(I)cc2F)c1NS(=O)(=O)C1(CC(O)CO)CC1]
wherein the 2-OH carbon is in the S configuration together with Sorafenib.

7. The use according to claims 1 to 5 wherein the combination comprises the MEK protein kinase inhibitor having the following structure: 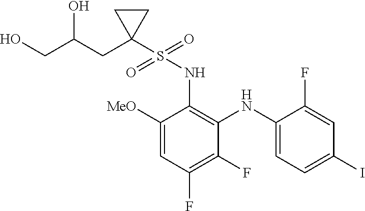 [SMILES: COc1cc(F)c(F)c(Nc2ccc(I)cc2F)c1NS(=O)(=O)C1(CC(O)CO)CC1]
and wherein the 2-OH carbon is in the S configuration together with Regonaferib.

8. Use of one or more biomarkers defined as mutated RAS for predicting the pharmaceutical efficacy or clinical response of at least one MEK protein kinase inhibitor to be administered to a HCC patient.

9. Use of one or more biomarkers defined as mutated RAS for predicting the pharmaceutical efficacy or clinical response of Sorafenib or Regorafenib to be administered to a HCC patient.

10. An in-vitro method comprising the step of
Identifying mutated-type RAS gene and/or protein in a test sample obtained from a HCC patient, characterized in that the method is for predicting the pharmaceutical efficacy or clinical response of a combination comprising a MEK protein kinase inhibitor and/or Sorafenib or Regorafenib to be administered to a HCC patient.

11. A kit comprising a suitable means for detecting mutated-type RAS gene or protein, for identifying biomarker defined as a mutated-type RAS, characterized in that the kit is for predicting the pharmaceutical efficacy or clinical response of a combination comprising a MEK protein kinase inhibitor and Sorafenib or Regorafenib to be administered to a HCC patient.

12. Use of a compound of formula A according to any one of claims 5, 6 or 7, for the preparation of a medicament for the treatment of hepatocellular carcinoma in a patient possessing a mutated KRAS, NRAS or HRAS gene.
